(19) United States
(12) Patent Application Publication
(10) Pub. No.: US 2016/0325480 A1
(43) Pub. Date: Nov. 10, 2016

(54) SELF-DIAGNOSTIC GRAFT PRODUCTION SYSTEMS AND RELATED METHODS

(71) Applicant: NEOGRAFT TECHNOLOGIES, INC., Taunton, MA (US)

(72) Inventors: Lorenzo SOLETTI, Pittsburgh, PA (US); Jon MCGRATH, Duxbury, MA (US); J. Christopher FLAHERTY, Auburndale, FL (US)

(21) Appl. No.: 15/108,970

(22) PCT Filed: Dec. 30, 2014
(86) PCT No.: PCT/US2014/072773

§ 371 Date: Jun. 29, 2016

Related U.S. Application Data
(60) Provisional application No. 61/922,545, filed on Dec. 31, 2013.

(51) Int. Cl. B29C 47/92; B29D 23/00; A61F 2/06

(52) U.S. Cl. CPC B29C 47/92; A61F 2/06; B29D 23/00; A61F 2240/001

(57) ABSTRACT

In some aspects, a system for producing a graft device can include a rotating assembly, a polymer delivery assembly, a controller and a diagnostic assembly. The rotating assembly can be constructed and arranged to rotate a tubular conduit. The polymer delivery assembly can be constructed and arranged to receive a polymer and deliver a fiber matrix comprising the polymer about the tubular conduit. The controller can be constructed and arranged to control the polymer delivery assembly and the rotating assembly. The diagnostic assembly can be constructed and arranged to detect an undesired state of at least one of the system or the graft device. Methods for producing a graft device are also provided.

### RELATED APPLICATIONS

This application claims the benefit of U.S. Provisional Application No. 61/922,545, filed Dec. 31, 2013, the contents of which are hereby incorporated herein by reference in their entirety.

This application is further related to U.S. patent application Ser. No. 13/502,759, filed Apr. 19, 2012; U.S. patent application Ser. No. 13/979,243, filed Jul. 11, 2013; U.S. patent application Ser. No. 14/364,989, filed Jun. 12, 2014; U.S. patent application Ser. No. 14/364,989, filed Jun. 12, 2014; International Patent Application Ser. No. PCT/US2014/056371, filed Sep. 18, 2014; International Patent Application Ser. No. PCT/US2014/065839, filed Nov. 14, 2014; U.S. patent application Ser. No. 13/515,996, filed Jun. 14, 2012; U.S. patent application Ser. No. 13/811,206, filed Jan. 18, 2013; U.S. patent application Ser. No. 13/984,249, filed Aug. 7, 2013; the contents of each of which are incorporated herein by reference in their entirety.

### TECHNICAL FIELD

This disclosure relates generally to systems for producing graft devices for a mammalian patient, and more particularly to systems for producing graft devices for providing cardiovascular bypass.

### BACKGROUND

Coronary artery disease, leading to myocardial infarction and ischemia, is currently a leading cause of morbidity and mortality worldwide. Current treatment alternatives consist of percutaneous transluminal angioplasty, stenting, and coronary artery bypass grafting (CABG). CABG can be carried out using either arterial or venous conduits and is the most effective and most widely used treatment to combat coronary arterial stenosis, with nearly 500,000 procedures being performed annually. In addition, there are approximately 80,000 lower extremity bypass surgeries performed annually. The venous conduit used for bypass procedures is most frequently the autogenous saphenous vein and remains the graft of choice for 95% of surgeons performing these bypass procedures. According to the American Heart Association, in 2004 there were 427,000 bypass procedures performed in 249,000 patients. The long term outcome of these procedures is limited due to occlusion of the graft vein or anastomotic site as a result of intimal hyperplasia (IH), which can occur over a timeframe of months to years.

Development of successful small diameter synthetic or tissue engineered vascular grafts has yet to be accomplished and use of arterial grafts (internal mammary, radial, or gastroepiploic arteries, for example) is limited by the short size, small diameter and availability of these veins. Despite their wide use, failure of arterial vein grafts (AVGs) remains a major problem: 12% to 27% of AVGs become occluded in the first year with a subsequent annual occlusive rate of 2% to 4%. Patients with failed AVGs usually require clinical intervention such as an additional surgery.

IH accounts for 20% to 40% of all AVG failures within the first 5 years after CABG surgery. Several studies have determined that IH develops, to some extent, in all mature AVGs and this development is regarded by many as an unavoidable response of the vein to grafting. IH is characterized by phenotypic modulation, followed by de-adhesion and migration of medial and adventitial smooth muscle cells (SMCs) and myofibroblasts into the intima where they proliferate. In many cases, this response can lead to stenosis and diminished blood flow through the graft. It is thought that IH may be initiated by the abrupt exposure of the veins to the dynamic mechanical environment of the arterial circulation.

### SUMMARY

For these and other reasons, there is a general need for systems, methods and devices that can provide enhanced AVGs and other improved grafts for mammalian patients. Desirably, the systems, methods, and devices will improve long term patency and minimize surgical and device complications such as those caused by improper or inadequate production of a graft device.

Embodiments of the systems and methods described herein can be directed to systems for producing graft devices for mammalian patients, as well as to methods for producing these graft devices.

According to an aspect of the technology described herein, systems for producing a graft device can include a rotating assembly, a polymer delivery assembly, a controller, and/or a diagnostic assembly. The rotating assembly can be constructed and arranged to rotate a tubular conduit. The polymer delivery assembly can be constructed and arranged to receive a polymer and deliver a fiber matrix comprising the polymer about the tubular conduit. The controller can be constructed and arranged to control the polymer delivery assembly and the rotating assembly. The diagnostic assembly can be constructed and arranged to detect an undesired state of at least one of the system or the graft device.

In some embodiments, the system can include an electrospinning system.

In some embodiments, the system is constructed and arranged to correct the detected undesired state.

In some embodiments, the system further comprises an alarm assembly constructed and arranged to activate when the undesired state is detected by the diagnostic assembly. The alarm assembly can comprise an alert selected from the group consisting of: audible alert; visual alert; tactile alert; and combinations of one or more of these or other alerts.

In some embodiments, the diagnostic assembly is constructed and arranged to detect an undesired state of a polymer delivery assembly parameter. The polymer delivery assembly parameter can represent the presence of a leak. The polymer delivery assembly parameter can represent a polymer flow rate. The system can further comprise a polymer flow pathway, and the polymer delivery assembly parameter can represent a level of undesired material in the polymer flow pathway. The undesired material can comprise undesired particulate. The undesired material can comprise material with an undesired homogeneity. The undesired material can comprise a gas bubble. The undesired material can comprise a material selected from the group consisting of: water; blood; lubricant; isopropyl alcohol; disinfectant; solvent; and combinations of one or more of these or other materials. The polymer can comprise an expiration date, and the polymer delivery assembly parameter can represent an expiration date of the polymer. The polymer can comprise a polymer parameter, and the polymer delivery assembly parameter can represent a polymer parameter selected from the group consisting of: polymer viscosity; polymer conductivity; polymer surface tension; polymer color; polymer turbidity; polymer chemical composition; polymer molecular weight profile; polymer magnetism; polymer impedance; and combinations of one or more of these or other polymer parameters. The polymer delivery assembly can comprise a nozzle constructed and arranged to translate, and the polymer delivery assembly parameter can represent the translation rate of the nozzle. The polymer delivery assembly can comprise a nozzle constructed and arranged to translate, and the polymer delivery assembly parameter can represent the translation acceleration of the nozzle. The polymer delivery assembly can comprise a nozzle constructed and arranged to translate, and the polymer delivery assembly parameter can represent the position of the nozzle. The polymer delivery assembly can comprise a nozzle constructed and arranged to translate, and the polymer delivery assembly parameter can represent the position of the nozzle relative to the rotating assembly. The polymer delivery assembly can comprise a nozzle constructed and arranged to translate, and the polymer delivery assembly parameter can represent nozzle vibration level. The polymer delivery assembly can comprise a nozzle constructed and arranged to translate, and the polymer delivery assembly parameter can represent status of a nozzle contacting an undesired object. The polymer delivery assembly parameter can represent a fiber parameter. The fiber parameter can comprise a fiber parameter selected from the group consisting of diameter; average diameter; diameter range; porosity; nodal density; alignment; flatness; twist; elasticity; crystallinity; conformity to target; water content; and combinations of one or more of these or other parameters. The polymer delivery assembly parameter can represent a fiber flight pathway parameter. The polymer delivery assembly parameter can represent a fiber matrix parameter. The fiber matrix parameter can comprise a fiber matrix parameter selected from the group consisting of: porosity; thickness; density; thickness distribution along the two longitudinal and circumferential axes; and combinations of one or more of these or other parameters. The polymer delivery assembly can comprise a nozzle, and the polymer delivery assembly parameter can represent a voltage level applied to the nozzle. The rotating assembly can comprise a mandrel constructed and arranged to be slidingly received by the tubular conduit, and the polymer delivery assembly parameter can further represent a voltage level applied to the mandrel. The polymer delivery assembly can comprise a nozzle, and the polymer delivery assembly parameter can represent the presence of icicles about the nozzle.

In some embodiments, the diagnostic assembly is constructed and arranged to detect an undesired state of a rotating assembly parameter. The rotating assembly can comprise a mandrel, and the rotating assembly parameter can represent rotational velocity of the mandrel. The rotating assembly can comprise a mandrel, and the rotating assembly parameter can comprise a voltage level applied to the mandrel. The rotating assembly can comprise a mandrel, and the rotating assembly parameter can represent alignment of the mandrel.

In some embodiments, diagnostic assembly is constructed and arranged to detect an undesired state of a controller parameter. The controller can comprise a power supply and the controller parameter can represent an input level of the power supply. The controller can comprise a power supply and the controller parameter can represent an output level of the power supply. The controller can comprise at least one electrical connection and the controller parameter can represent connection status of the at least one electrical connection.

In some embodiments, the diagnostic assembly is constructed and arranged to detect an undesired state of a tubular conduit parameter. The tubular conduit parameter can represent a diameter of the tubular conduit. The tubular conduit parameter can represent level of trauma in the tubular conduit. The tubular conduit can comprise a wall, and the level of trauma can represent a level of disruption in the wall of the tubular conduit. The tubular conduit can comprise a wall, and the tubular conduit parameter can represent the status of a leak in the wall of the tubular conduit. The leak can comprise a leak in an insufficiently ligated side branch of the tubular conduit.

In some embodiments, the diagnostic assembly is constructed and arranged to detect an undesired state of a fiber matrix parameter. The fiber matrix parameter can represent a thickness of the fiber matrix. The fiber matrix parameter can represent a dryness level of the fiber matrix. The fiber matrix parameter can represent a fiber matrix parameter selected from the group consisting of: fiber diameter; fiber average diameter; fiber diameter range; nodal density; fiber alignment; fiber flatness; fiber twist; fiber elasticity; fiber crystallinity; fiber conformity to target; fiber water content; fiber matrix porosity; fiber matrix thickness; fiber matrix density;

fiber matrix thickness distribution along the longitudinal and circumferential axes; and combinations of one or more of these or other parameters.

In some embodiments, the graft device further comprises one or more spines, and the diagnostic assembly can be constructed and arranged to detect an undesired state of a spine parameter. The spine parameter can represent the position of the spine about the tubular conduit. The spine parameter can comprise a spine parameter selected from the group consisting of: spine size; spine position; compression level applied to tubular conduit; and combinations of one or more of these or other parameters.

In some embodiments, the diagnostic assembly is constructed and arranged to detect an undesired state of a graft device parameter. The diagnostic assembly can be constructed and arranged to detect an undesired state of a graft device parameter. The graft device parameter can represent a solvent level present in the graft device.

In some embodiments, the system comprises a sensor constructed and arranged to collect data used to detect the undesired state of that at least one of the system or the graft device. The sensor can comprise a sensor selected from the group consisting of: environmental sensor; pressure sensor; strain gauge; temperature sensor; humidity sensor; vibration sensor; pH sensor; chemical sensor; solvent sensor; magnetic sensor; electromagnetic sensor; ultrasonic sensor; flow sensor; viscosity sensor; visual sensor; optical sensor; light sensor; and combinations of one or more of these or other sensors. The sensor can comprise a viscosity sensor. The data collected can comprise polymer viscosity data. The system can further comprise an environmental chamber surrounding at least a portion of the rotating assembly, and the sensor can comprise an environmental parameter sensor constructed and arranged to measure an environmental parameter within the environmental chamber. The measured environmental parameter can comprise a parameter selected from the group consisting of: temperature; humidity; pressure; and combinations of one or more of these or other parameters. The sensor can comprise a temperature sensor. The system can further comprise a polymer storage device, and the data produced by the temperature sensor can represent a thermal history of the polymer storage device. The temperature sensor can be constructed and arranged to measure temperature of the polymer. The system can be constructed and arranged to filter the polymer, and the temperature sensor can be constructed and arranged to measure the temperature of the polymer during filtration. The sensor can comprise a leak-detecting sensor. The leak sensor can comprise a fluid-detecting sensor. The leak sensor can comprise a pressure sensor. The sensor can comprise a polymer solution homogeneity sensor. The polymer solution homogeneity sensor can comprise a light sensor. The sensor can comprise at least one of a motion sensor or a position sensor. The at least one of a motion sensor or a position sensor can comprise a sensor selected from the group consisting of: optical; magnetic; and combinations of one or more of these sensors. The at least one of a motion sensor or a position sensor can be constructed and arranged to detect an undesired translation of the polymer delivery assembly. The rotating assembly can further comprise a mandrel, and the at least one of a motion sensor or a position sensor can be constructed and arranged to detect undesired rotation of the mandrel. The sensor can comprise a voltage sensor. The voltage sensor can be constructed and arranged to detect voltage of the polymer delivery assembly. The polymer delivery assembly can comprise a nozzle, and the voltage sensor can be constructed and arranged to detect voltage of the nozzle. The rotating assembly can comprise a mandrel, and the voltage sensor can be constructed and arranged to detect voltage of the mandrel. The sensor can comprise an image producing sensor. The image producing sensor can comprise a camera. The system can further comprise an image processing algorithm constructed and arranged to analyze the data produced by the image producing sensor, and the detection of the undesired state can be based on the analysis. The polymer delivery assembly can comprise a nozzle, and the image producing sensor can be constructed and arranged to provide visual information related to fibers delivered by the nozzle. The nozzle can be constructed and arranged to produce a Taylor Cone proximate the nozzle tip, and the image producing sensor can be constructed and arranged to provide visual information related to the Taylor Cone. The image producing sensor can be constructed and arranged to provide visual information related to a fiber parameter selected from the group consisting of: fiber transparency; fiber translucency; fiber diameter; and combinations of one or more of these or other parameters. The image producing sensor can be constructed and arranged to provide visual information related to any undesired objects proximate the nozzle. The sensor can comprise a measurement sensor. The measurement sensor can comprise a visual sensor. The visual sensor can comprise a camera. The measurement sensor can comprise an optical sensor. The optical sensor can comprise a laser. The measurement sensor can comprise a surface-detecting sensor. The surface-detecting sensor can comprise a sensor selected from the group consisting of: light sensor; radar sensor; sonar sensor; and combinations of one or more of these or other sensors. The sensor can be constructed and arranged to measure a fiber matrix property. The fiber matrix property can comprise a fiber matrix property selected from the group consisting of: fiber diameter; fiber average diameter; fiber diameter range; nodal density; fiber alignment; fiber flatness; fiber twist; fiber elasticity; fiber crystallinity; fiber conformity to target; fiber water content; fiber matrix porosity; fiber matrix thickness; fiber matrix density; fiber matrix thickness distribution along the longitudinal and circumferential axes; and combinations of one or more of these or other properties. The sensor can comprise a flow sensor. The flow sensor can be constructed and arranged to measure a polymer flow rate. The system can further comprise an environmental chamber surrounding at least a portion of the rotating assembly, and the flow sensor can be constructed and arranged to measure the flow rate of gas supplied to the environmental chamber. The system can further comprise an environmental chamber surrounding at least a portion of the rotating assembly, and the flow sensor can be constructed and arranged to measure the flow rate of gas evacuated from the environmental chamber. The system can further comprise an environmental control chamber surrounding at least a portion of the polymer delivery assembly, and the flow sensor can be constructed and arranged to measure the flow rate of a gas within the environmental control chamber. The polymer delivery assembly can comprise a nozzle, and the flow sensor can be constructed and arranged to measure the flow rate into the nozzle. The sensor can comprise an occlusion sensor. The system can further comprise at least one polymer flow pathway and the occlusion sensor can be constructed and arranged to measure flow in the at least one polymer flow pathway. The sensor can comprise a sensor constructed and arranged to measure a contamination level. The system can further comprise at least one polymer flow pathway and the contamination sensor can be constructed and arranged to measure contamination level in the at least one polymer flow pathway. The contamination sensor can be constructed and arranged to measure contamination level in and/or on the fiber matrix. The contamination sensor can be constructed and arranged to measure contamination level in and/or on the tubular conduit. The sensor can comprise a sensor constructed and arranged to measure a level of solvent. The sensor can comprise a sensor selected from the group consisting of: colorimetric detector tube; passive (diffusion) badge dosimeter; sorbent tube sampling device; combustible gas monitor such as a monitoring using a hot bead or a hot wire; combustible gas sensor; photoionization detector; flame ionization detector; infrared spectra-photometer; and combinations of one or more of these or other sensors. The system can further comprise an environmental chamber surrounding at least a portion of the rotating assembly and a filter on an outflow port of the environmental chamber, and the sensor can be constructed and arranged to measure a parameter of the outflow port filter. The sensor can be constructed and arranged to measure a parameter selected from the group consisting of: weight of the outflow port filter; flow through the outflow port filter; and combinations of one or more of these or other parameters.

In some embodiments, the system further comprises an information element and an information element reader device constructed and arranged to collect data from the information element, and the diagnostic assembly analyzes the collected data to detect the undesired state of at least one of the system or the graft device. The information element can comprise an element selected from the group consisting of: barcode; microchip; RFID; and combinations of one or more of these or other elements. The system can further comprise a polymer storage device comprising the information element, and the information element data can comprise polymer data. The diagnostic assembly can detect the applicability of the polymer based on the polymer data. The diagnostic assembly can detect an expiration date of the polymer based on the polymer data.

In some embodiments, the system further comprises a timer constructed and arranged to measure the time period of delivery of the fiber matrix to the tubular conduit. The undesired state detected by the diagnostic assembly can comprise a measured time period of delivery below a minimum. The undesired state detected by the diagnostic assembly can comprise a measured time period of delivery above a maximum.

According to another aspect, a method of producing a graft device comprises selecting a system as described herein, and applying a fiber matrix about a tubular conduit.

In some embodiments, the method further comprises entering an alarm state when an undesired state of at least one of the system or graft device is detected. Entering the alarm state can comprise producing an alert signal. The alert signal can comprise a signal selected from the group consisting of: audible alert; visual alert; tactile alert; and combinations of one or more of these or other signals. Entering the alarm state can comprise stopping the delivery of the fiber matrix about the tubular conduit.

### BRIEF DESCRIPTION OF THE DRAWINGS

The foregoing and other objects, features and advantages of embodiments of the technology described herein will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view of an example system for producing a graft device.

FIG. 2 is a partial cutaway view of an example graft device.

FIG. 2A is a sectional view of an example embodiment of the graft device of FIG. 1, comprising a tubular conduit and a surrounding fiber matrix.

FIG. 2B is a sectional view of another example embodiment of the graft device of FIG. 1, comprising a tubular conduit, a spine and a surrounding fiber matrix.

FIG. 3 is a schematic view of an example system for producing a graft device with an electrospun fiber matrix.

FIG. 4 is a side sectional view of a portion of the electrospinning device of FIG. 3.

### DETAILED DESCRIPTION

The terminology used herein is generally for the purpose of describing certain embodiments and is not intended to be limiting of the inventive concepts. Furthermore, embodiments described herein may include several novel features, no single one of which is solely responsible for its desirable attributes or which is essential to practicing the concepts described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

Provided herein are systems and methods for producing graft devices for implantation in a mammalian patient, such as to carry fluids (e.g. blood or other body fluid) from a first anatomical location to a second anatomical location. The systems described herein can include a diagnostic assembly constructed and arranged to detect an undesired state of the system and/or an undesired state of a graft device being produced by the system. The graft devices include a tubular conduit (e.g. a harvested blood vessel segment, other harvested tissue and/or an artificial conduit) and a fiber matrix that surrounds the tubular conduit. The fiber matrix is typically applied with one or more of: an electrospinning device; a melt-spinning device; a melt-electrospinning device; a misting assembly; a sprayer; an electrosprayer; a fuse deposition device; a selective laser sintering device; a three-dimensional printer; or other fiber matrix delivery device. The fiber matrix delivery process can be performed in an operating room, such as when the tubular conduit is a harvested saphenous vein segment to be anastomosed between the aorta and a location on a diseased coronary artery distal to an occlusion. In these cardiovascular bypass procedures, end to side anastomotic connections are typically used to attach the graft device to the aorta and the diseased artery. Alternatively, a side to side anastomosis can be used, such as to attach an end of the graft device to multiple arteries in a serial fashion.

The fiber matrix can comprise one or more materials, such as one or more similar or dissimilar polymers as described in detail herebelow. The fiber matrix can comprise a biodegradable, bioerodible or bioabsorbable (hereinafter “biodegradable”) material or otherwise be configured such that the support to the graft device provided by the fiber matrix changes over time after implantation. Numerous biodegradable polymers can be used such as: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations of these. Dunn et al. (U.S. Pat. No. 4,655,777) discloses a medical implant including bioabsorbable fibers that reinforce a bioabsorbable polymer matrix. Alternatively or additionally, the fiber matrix can comprise one or more portions including durable or otherwise non-biodegradable materials configured to remain intact for long periods of time when implanted, such as at least 6 months or at least 1 year.

The graft devices can further include one or more spines or other kink resisting elements (hereinafter “spine”), such as to prevent luminal narrowing, radial collapse, kinking and/or other undesired movement of the graft device (e.g. movement into an undesired geometric configuration), such as movement that occurs while implanting the graft device during a surgical procedure and/or at a time after implantation. One or more spine can be placed inside the tubular conduit, between the tubular conduit and the fiber matrix, between layers or within layers of the fiber matrix and/or outside the fiber matrix. The spine can comprise a biodegradable material or otherwise be configured to provide a temporary support to the graft device. Alternatively or additionally, the spine can comprise one or more portions including durable or otherwise non-biodegradable materials configured to remain intact for long periods of time when implanted, such as at least 6 months or at least 1 year.

The systems described herein typically include an electrospinning device and/or other fiber or fiber matrix delivery assembly. In some embodiments, the graft device further comprises a spine or other kink resisting element. The spine can comprise a component that is applied, placed and/or inserted, such as by the fiber matrix delivery assembly (e.g. automatically or semi-automatically) or with a placement or insertion tool (e.g. manually).

The graft devices described herein can include an electrospun fiber matrix such as those disclosed in U.S. patent application Ser. No. 13/502,759, filed Apr. 19, 2012, the content of which are incorporated herein by reference in their entirety. The technology described herein can include graft devices, as well as systems, tools and methods for producing and/or implanting graft devices, such as those disclosed in applicant's co-pending applications U.S. patent application Ser. No. 13/515,996, filed Jun. 14, 2012; U.S. patent application Ser. No. 13/811,206, filed Jan. 18, 2013; U.S. patent application Ser. No. 13/979,243, filed Jul. 11, 2013; U.S. patent application Ser. No. 13/984,249, filed Aug. 7, 2013; U.S. patent application Ser. No. 14/354,025, filed Apr. 24, 2014; U.S. patent application Ser. No. 14/378,263, filed Aug. 12, 2014; the contents of each of which are incorporated herein by reference in their entirety.

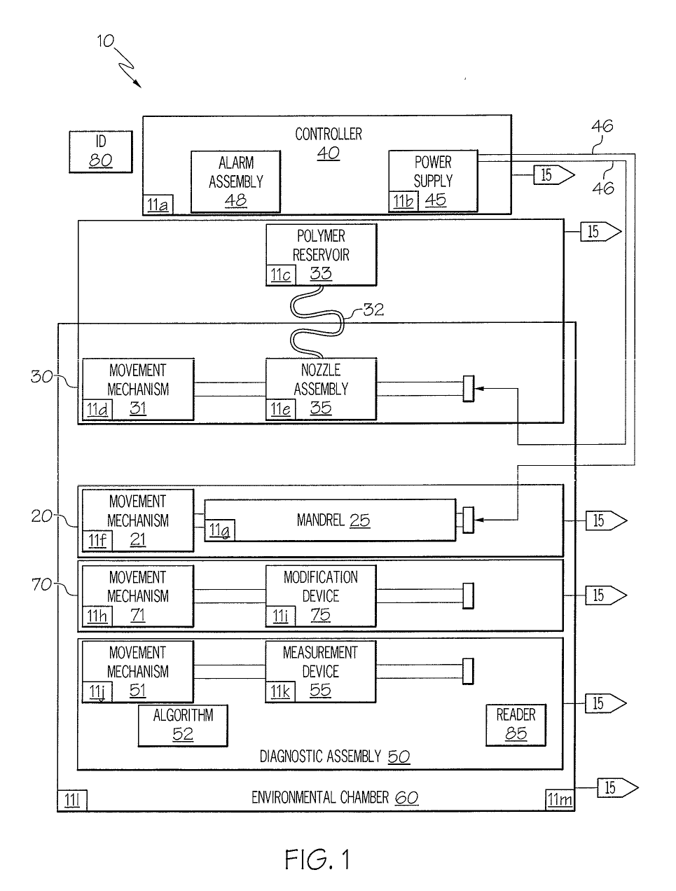

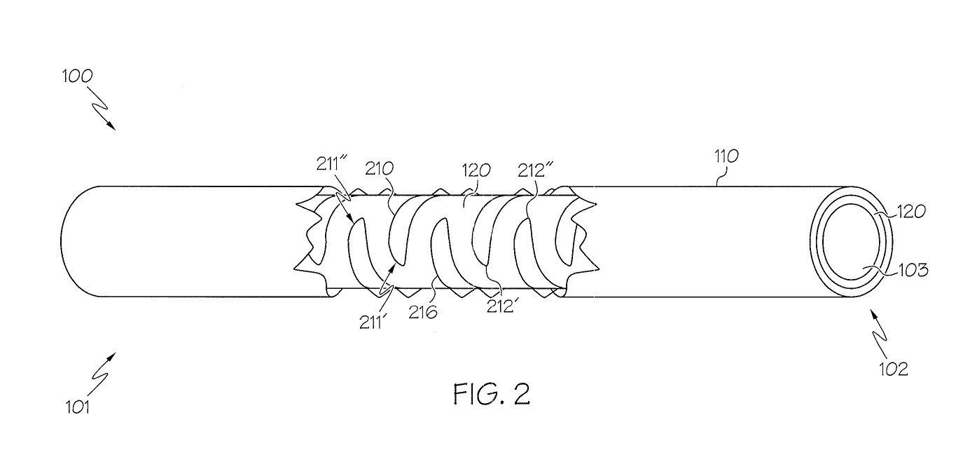

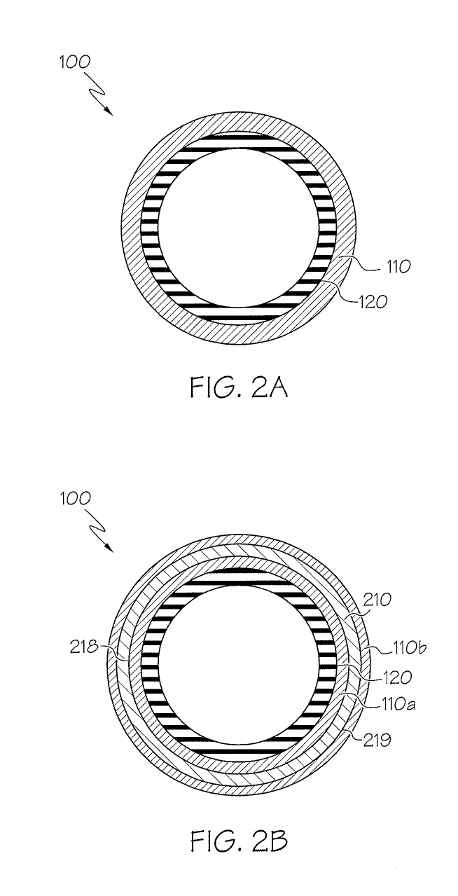

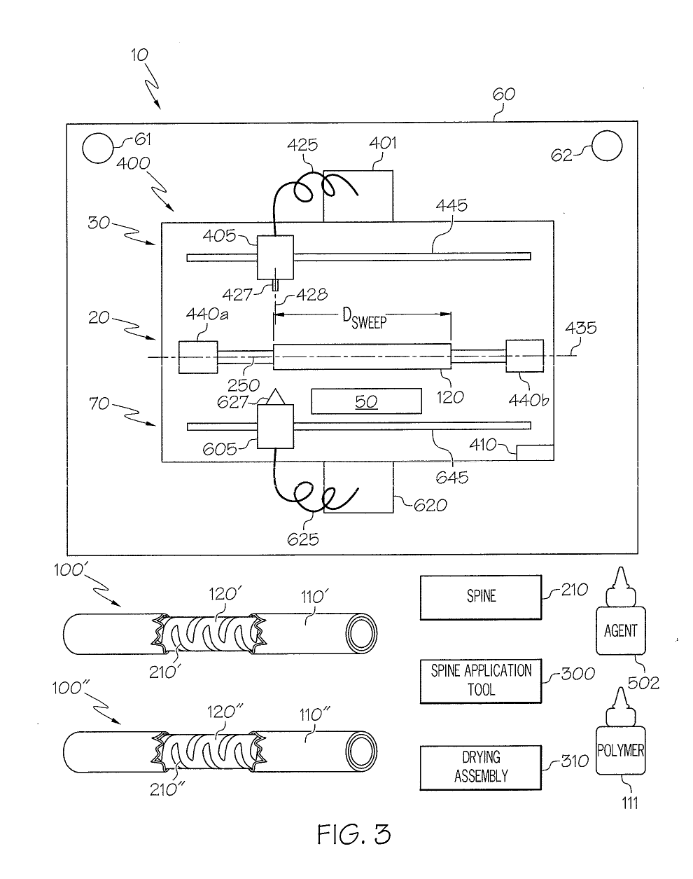

Referring now to FIG. 1, a schematic view of an example system for producing a graft device is illustrated. System 10 includes various components and assemblies (hereinafter “components”) used to create a graft device for a mammalian patient, such as a graft device 100 described herebelow in reference to FIGS. 2, 2A, 2B, or 3. System 10 of FIG. 1 includes rotating assembly 20, polymer delivery assembly 30, controller 40 and diagnostic assembly 50. In some embodiments, system 10 comprises an electrospinning system configured to deliver a fiber matrix about a tubular conduit. In some embodiments, system 10 is constructed and arranged similar to system 10 and/or rotating assembly 20 of FIG. 3 described herebelow. Rotating assembly 20 can include mandrel 25 which is constructed and arranged to slidingly receive a tubular conduit such as an artificial or living tissue conduit as described herein. Rotating assembly 20 can include a rotational drive assembly, movement mechanism 21 which is constructed and arranged to rotate mandrel 25 and any tubular conduit attached thereto. In some embodiments, rotating assembly 20 and/or mandrel 25 are constructed and arranged similar to rotating assembly 20 and/or mandrel 250, respectively, of FIG. 3, described herebelow.

Polymer delivery assembly 30 includes nozzle assembly 35. Nozzle assembly 35 is attached to polymer reservoir 33 via tubing 32. Polymer reservoir 33 can comprise a pumping element, such as a syringe or peristaltic pumping element configured to deliver one or more polymers or polymer solutions (e.g. a liquid comprising one or more polymers dissolved in a solvent) to nozzle assembly 35 via tubing 32. Nozzle assembly 35 is constructed and arranged to at least translate (e.g. to reciprocally translate) via movement mechanism 31. In some embodiments, nozzle assembly 35 is constructed and arranged to be rotated via movement mechanism 31 (e.g. rotate about the central axis of mandrel 25 such as when mandrel 25 does not rotate). Polymer delivery assembly 30 is constructed and arranged to receive polymer from polymer reservoir 33 and deliver a fiber matrix about a tubular conduit positioned on a portion of rotating assembly 20. Polymer delivery assembly 30 and/or one or more of its components can be constructed and arranged similar to the similar components of polymer delivery assembly 30 of FIG. 3 described herebelow.

System 10 and other similar systems typically include diagnostic assembly 50 which is constructed and arranged to detect an undesired state of at least one of system 10 and/or a graft device produced or being produced by system 10, such as a graft device 100 of FIGS. 2, 2A, 2B, or 3. Diagnostic assembly 50 can comprise one or more sensors or sensor assemblies (hereinafter “sensors”), such as measurement device 55 and/or sensor 11. An undesired state of system 10 and/or a graft device 100 can be detected based on information received from one or more sensors 11, such as information related to one or more parameters of system 10 and/or one or more parameters of a graft device 100, each as described in detail herebelow. Diagnostic assembly 50 can comprise one or more algorithms, such as algorithm 52 configured to receive signals from one or more assemblies, sensors and/or other components of system 10, such as the sensors 11a-11k described herebelow. The undesired state can be detected via algorithm 52 by mathematically processing and/or otherwise analyzing received information. The undesired state can be detected by comparing a parameter value to a threshold. The parameter value can be a current value for a system 10 or device 100 parameter or a mathematically calculated value such as an average, peak, minimum, maximum or other processed value. The undesired state can correlate to one or more parameter values “exceeding” a threshold, such as when a parameter value is above a maximum threshold, below a minimum threshold and/or outside a range of acceptable values.

Measurement device 55 can be constructed and arranged to measure one or more parameters such as one or more parameters of system 10 and/or graft device 100. In some embodiments, measurement device 55 is constructed and arranged to translate, rotate and/or otherwise move via movement mechanism 51. In some embodiments, movement mechanism 51 is synchronized with movement mechanism 31 of polymer delivery assembly 30 such that measurement device 55 moves in unison with nozzle assembly 35 or otherwise moves in a pattern related to the position and/or movement of nozzle assembly 35.

System 10 can include modification assembly 70 which can be constructed and arranged to modify the graft device produced by system 10 and/or otherwise perform a function on the graft device produced by system 10. Modification assembly 70 can include modification device 75. Modification device 75 can be translated, rotated and/or otherwise moved by movement mechanism 71. Modification assembly 70 and/or one or more of its components can be constructed and arranged similar to the similar components of modification assembly 70 of FIG. 3 described herebelow.

System 10 includes bus 15 which comprises one or more wires, optical fibers, cables and/or fluid conduits constructed and arranged to transmit one or more of electrical power, data, optical power, optical data, fluid (e.g. hydraulic and/or pneumatic fluid) among and/or between one or more components of system 10. Bus 15 can be operably connected to one or more assemblies selected from the group consisting of: controller 40; polymer delivery assembly 30; rotating assembly 20; diagnostic assembly 50; environmental chamber 60; modification assembly 70; and/or another component or assembly of system 10.

Controller 40 can be constructed and arranged to provide control signals and receive information signals, such as via bus 15. Controller 40 includes power supply 45 which is electrically connected to mandrel 25 and nozzle assembly 35 via conductors 46. Controller 40 is constructed and arranged to control polymer delivery assembly 30 (e.g. control the flow rate) and rotating assembly 20 (e.g. control the rotational velocity), such as via signals sent via bus 15.

As described above, system 10 can comprise one or more sensors, such as sensors 11a-11m. Sensors 11a-11m (generally sensor 11), can comprise a single sensor or multiple sensors. Sensors 11 can comprise one or more sensors selected from the group consisting of: environmental sensor; pressure sensor; strain gauge; temperature sensor; humidity sensor; vibration sensor; pH sensor; chemical sensor; solvent sensor; magnetic sensor; electromagnetic sensor; ultrasonic sensor; flow sensor; viscosity sensor; visual sensor; optical sensor; light sensor; and combinations thereof.

Controller 40 can include one or more sensors, such as sensor 11a and/or sensor 11b as shown. Sensor 11b can be constructed and arranged to measure one or more parameters of power supply 45, such as by being positioned in, on, within and/or otherwise proximate (hereinafter “proximate”) power supply 45.

Polymer delivery assembly 30 can comprise one or more sensors, such as sensors 11c, 11d, and/or 11e as shown. Sensor 11c can be constructed and arranged to measure one or more parameters of polymer reservoir 33. Sensor 11d can be constructed and arranged to measure one or more parameters of movement mechanism 31. Sensor 11e can be constructed and arranged to measure one or more parameters of nozzle assembly 35. In some embodiments, sensor 11c, sensor 11e and/or another sensor 11 of system 10 comprises a viscosity sensor, such as a sensor constructed and arranged to measure the viscosity of polymer delivered from polymer reservoir 33. In some embodiments, sensor 11e and/or another sensor 11 of system 10 comprises a flow rate sensor and/or a bubble detector sensor (e.g. an ultrasonic bubble detector sensor).

Rotating assembly 20 can comprise one or more sensors, such as sensors 11f and/or 11g as shown. Sensor 11f can be constructed and arranged to measure one or more parameters of movement mechanism 21. Sensor 11g can be constructed and arranged to measure one or more parameters of mandrel 25. In some embodiments, sensors 11f or 11g comprises a magnetic sensor, an accelerometer and/or a light sensor configured to measure movement of movement mechanism 21 and/or mandrel 25.

Modification assembly 70 can comprise one or more sensors, such as sensors 11h and/or 11i as shown. Sensor 11h can be constructed and arranged to measure one or more parameters of movement mechanism 71. Sensor 11i can be constructed and arranged to measure one or more parameters of modification device 75.

Diagnostic assembly 50 can comprise one or more sensors, such as sensors 11j and/or 11k as shown. Sensor 11j can be constructed and arranged to measure one or more parameters of movement mechanism 51, such as when sensor 11j comprises a magnetic sensor, an accelerometer and/or a light sensor configured to measure movement of movement mechanism 51. Sensor 11k can be constructed and arranged to measure one or more parameters of measurement device 55.

Environmental chamber 60 can comprise one or more sensors, such as sensors 11l and/or 11m as shown. Sensors 11l and/or 11m can be constructed and arranged to measure one or more parameters of environmental chamber 60, such as when sensor 11l is positioned proximate an inlet port of environmental chamber 60 and/or sensor 11m is positioned proximate an outlet port of environmental chamber 60, as described in reference to environmental chamber 60 of FIG. 3 herebelow. In some embodiments, sensor 11j, sensor 11k and/or another sensor of system 10 is constructed and arranged to measure an environmental parameter of environmental chamber 60, such as a parameter selected from the group consisting of: temperature; humidity; pressure; and combinations thereof.

In some embodiments, one or more sensors 11 comprise a temperature sensor. Sensor 11c, sensor 11e, and/or another sensor of system 10 can comprise a temperature sensor constructed and arranged to measure the temperature of polymer within system 10. For example, polymer reservoir 33 or another component of system 10 can comprise a filter constructed and arranged to filter polymer at an elevated temperature, and the sensor 11 can be configured to measure the temperature of the polymer during filtration. Alternatively or additionally, a temperature sensor 11 can be constructed and arranged to gather temperature history information such as a sensor 11 positioned on a polymer storage device configured to be inserted into or otherwise provide polymer to polymer reservoir 33.

In some embodiments, one or more sensors 11 comprise a leak-detecting sensor. Sensor 11c, sensor 11e, and/or another sensor of system 10 can comprise a leak-detecting sensor constructed and arranged to assess the status of one or more leaks within system 10. In these embodiments, a sensor 11 can comprise a fluid-detecting sensor and/or a pressure sensing sensor configured to detect a leak (e.g. a polymer leak from polymer reservoir 33, tubing 32 and/or nozzle assembly 35).

In some embodiments, one or more sensors 11 comprise a polymer solution homogeneity sensor. Sensor 11c, sensor 11e, and/or another sensor of system 10 can comprise a polymer solution homogeneity sensor constructed and arranged to assess the homogeneity level of a solution comprising one or more polymers, at one or more locations within system 10 (e.g. within polymer reservoir 33, tubing 32 and/or nozzle assembly 35). In these embodiments, a sensor 11 can comprise an optical or light sensor configured to assess solution homogeneity.

In some embodiments, one or more sensors 11 comprise a motion and/or position sensor. Sensor 11c, 11d, 11e, 11f, 11g, 11h, 11i, 11j, 11k, and/or another sensor of system 10 can comprise a motion and/or position sensor constructed and arranged to determine and/or assess the motion and/or position of one or more components of system 10, such as nozzle assembly 35, mandrel 25, modification device 75 and/or measurement device 55. In these embodiments, a sensor 11 can comprise a sensor selected from the group consisting of: optical sensor; magnetic sensor (e.g. a hall effect sensor); accelerometer; and combinations of these. The sensor 11 can be configured to provide information to detect an undesired translation of nozzle assembly 35, modification device 75 and/or measurement device 55. Alternatively or additionally, the sensor 11 can be configured to provide information to detect an undesired rotation of mandrel 25.

In some embodiments, one or more sensors 11 comprise a voltage sensor. Sensor 11b, 11e, 11g, and/or another sensor of system 10 can comprise a voltage sensor constructed and arranged to measure voltage of one or more components of system 10 such as nozzle assembly 35 and/or mandrel 25 (e.g. to determine if an undesired potential difference exists between a nozzle of nozzle assembly 35 and mandrel 25).

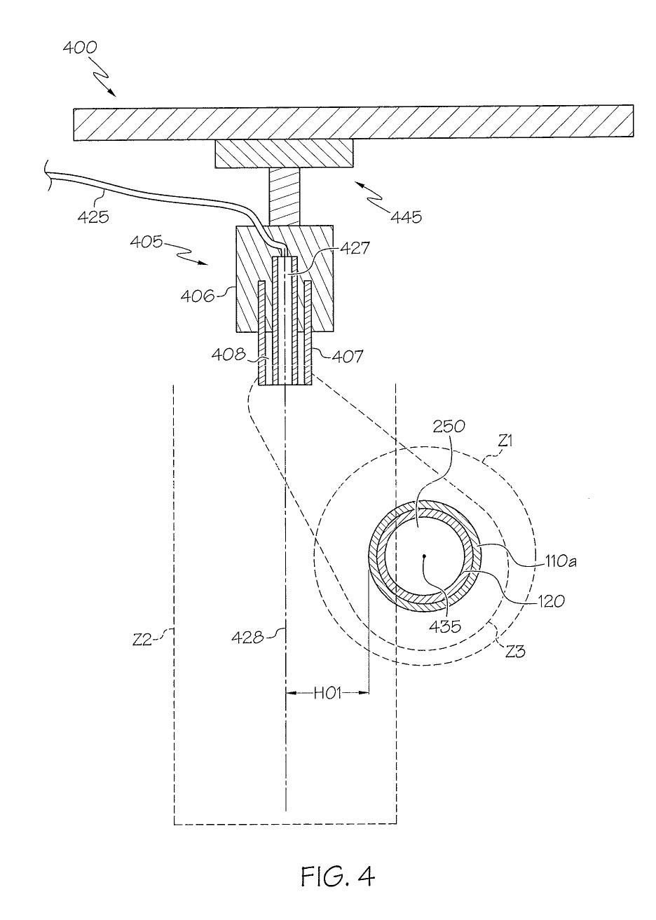

In some embodiments, one or more sensors 11 comprise an image producing sensor, such as a sensor 11 comprising a camera (e.g. a visual light or infrared camera). In these embodiments, algorithm 52 or another component of diagnostic assembly 50 can comprise an image processing algorithm. In these embodiments, the image producing sensor 11 can be configured to provide visual information related to the area proximate a nozzle of nozzle assembly 35 (e.g. by providing visual images of fibers delivered by the nozzle to algorithm 52). In some embodiments, the image producing sensor 11 provides visual information related to a Taylor Cone present at the nozzle tip, such as to detect an undesired shape of the Taylor cone. In some embodiments, the image producing sensor 11 provides visual information related to a fiber parameter selected from the group consisting of: fiber transparency; fiber translucency; fiber diameter; and combinations thereof. In some embodiments, the image producing sensor 11 provides visual information related to objects proximate nozzle assembly 35, such as to detect an undesired state in which an object proximate nozzle assembly 35 might adversely affect fiber delivery (as described below in reference to the “object free zone” described herebelow in reference to FIG. 4.

In some embodiments, one or more sensors 11 comprise a measurement sensor. In these embodiments, sensor 11 can comprise measurement device 55 which can be constructed and arranged to measure a graft device 100 parameter (e.g. a tubular conduit 120 parameter and/or a fiber matrix 110 parameter). In these embodiments, sensor 11 can comprise a visual sensor such as a camera configured to provide visual information from which measurement information can be extracted. Alternatively or additionally, sensor 11 can comprise an optical sensor (e.g. a laser micrometer or other laser-based measuring sensor) or a surface detecting sensor (e.g. a light sensor, radar sensor and/or a sonar sensor). A measurement sensor 11 can be constructed and arranged to measure a fiber matrix 110 property selected from the group consisting of: fiber diameter; fiber average diameter; fiber diameter range; nodal density; fiber alignment; fiber flatness; fiber twist; fiber elasticity; fiber crystallinity; fiber conformity to target; fiber water content; fiber matrix porosity; fiber matrix thickness; fiber matrix density; fiber matrix thickness distribution along the longitudinal and circumferential axes; and combinations of one or more of these or other properties.

In some embodiments, one or more sensors 11 comprise a flow sensor. Flow sensor 11c, 11e, and/or another sensor 11 of system 10 can comprise a flow sensor constructed and arranged to measure a polymer flow rate, such as a polymer flow rate into a nozzle of nozzle assembly 35. Sensor 11l, 11m, and/or another sensor 11 of system 10 can comprise a flow sensor constructed and arranged to measure the flow rate of a gas, such as a flow of gas supplied to environmental chamber 60, a flow of gas evacuated from environmental chamber 60 and/or a flow of gas present within environmental chamber 60.

In some embodiments, one or more sensors 11 comprise an occlusion sensor. Sensor 11c, 11e, and/or another sensor of system 10 can comprise an occlusion sensor constructed and arranged to measure occlusion of flow within one or more flow pathways of system 10, such as tubing 32.

In some embodiments, one or more sensors 11 comprise a contamination sensor. Sensor 11c, 11e, and/or another sensor of system 10 can comprise a contamination sensor such as a contamination sensor that is constructed and arranged to detect contamination level in a polymer storage or delivery location such as polymer reservoir 33, tubing 32, and/or nozzle assembly 35. Alternatively or additionally, a sensor 11 can be constructed and arranged to measure a contamination level on fiber matrix 110, and/or tubular conduit 120.

In some embodiments, one or more sensors 11 comprise a solvent level sensor. In these embodiments, the solvent level sensor can comprise a sensor selected from the group consisting of: colorimetric detector tube; passive (diffusion) badge dosimeter; sorbent tube sampling device; combustible gas monitor such as a monitoring using a hot bead or a hot wire; combustible gas sensor; photoionization detector; flame ionization detector; infrared spectra-photometer; and combinations of these or other sensors. Alternatively or additionally, the sensor 11 can comprise a sensor configured to measure an outflow parameter of environmental chamber 60, such as when the outflow is filtered and the sensor 11 measures the weight of the filter (e.g. when weight above a threshold correlates to an undesired level of solvent present) and/or measures the flow the filter (e.g. when flow below a threshold correlates to an undesired level of solvent present).

In some embodiments, diagnostic assembly 50 is constructed and arranged to monitor one or more polymer delivery assembly 30 parameters, such as to detect an undesired state of one or more of those parameters. For example, the undesired state correlates to one or more of these polymer delivery assembly 30 parameters exceeding a threshold, such as are described hereabove. In some embodiments, the polymer delivery assembly 30 parameter can represent a polymer flow rate or the presence of a leak, such as a leak from polymer reservoir 33, tubing 32, and/or nozzle assembly 35. Alternatively or additionally, the monitored polymer delivery assembly 30 parameter can represent a level of undesired material (e.g. a solid particulate or a gas bubble) in the polymer or the polymer flow pathway, where the undesired state correlates to an amount of undesired material above a maximum threshold (e.g. a threshold of zero detected by absorption of light or other means). The parameter can represent the level of homogeneity of a polymer solution, where the undesired state correlates to improper mixing or undesired settling of polymer. The parameter can represent the level of one or more contaminants in a polymer solution, such as a contaminant selected from the group consisting of: water; blood; lubricant; isopropyl alcohol; disinfectant; solvent; and combinations of one or more of these or other contaminants.

In some embodiments, a polymer delivery assembly 30 parameter monitored by diagnostic assembly 50 can represent a polymer parameter, such as a polymer parameter selected from the group consisting of: polymer viscosity; polymer conductivity; polymer surface tension; polymer color; polymer turbidity; polymer chemical composition; polymer molecular weight profile; polymer magnetism; polymer impedance; and combinations of one or more of these or other parameters. In some embodiments, the polymer delivery assembly 30 parameter monitored comprises a nozzle assembly 35 parameter, such as translation rate, translation acceleration, position (e.g. position relative to a component of rotating assembly 20), vibration level, or contact of nozzle assembly 35 with an undesired object.

In some embodiments, a polymer delivery assembly 30 parameter monitored by diagnostic assembly 50 can represent a fiber parameter, such as a fiber parameter selected from the group consisting of: diameter; average diameter; diameter range; porosity; nodal density; alignment; flatness; twist; elasticity; crystallinity; conformity to target; water content; and combinations of one or more of these or other parameters. Alternatively or additionally, the monitored parameter can represent a condition of the fiber flight pathway (e.g. by monitoring the spatial dynamics of fibers emanating from the nozzle), such as to detect an undesired location for a fiber being delivered.

In some embodiments, a polymer delivery assembly 30 parameter monitored by diagnostic assembly 50 can represent a fiber matrix parameter, such as a fiber matrix parameter selected from the group consisting of: porosity; thickness; density; thickness distribution along the two longitudinal and circumferential axes; and combinations of one or more of these or other parameters.

In some embodiments, a polymer delivery assembly 30 parameter monitored by diagnostic assembly 50 can represent the voltage level applied to a nozzle of nozzle assembly 35 (e.g. potential difference between a nozzle of nozzle assembly 35 and mandrel 25). In some embodiments, the monitored parameter can represent the presence of and/or amount of “icicles” about a nozzle of nozzle assembly 35 (i.e. to detect an undesired amount of solidified or solidifying polymer solution lingering proximate to and/or attached to the nozzle).

In some embodiments, diagnostic assembly 50 is constructed and arranged to monitor one or more rotating assembly 20 parameters, such as to detect an undesired state of one or more of those parameters. For example, the undesired state correlates to one or more of these rotating assembly 20 parameters exceeding a threshold. In some embodiments, the rotating assembly 20 monitored parameter represents a rotational velocity of mandrel 25. In some embodiments, the monitored parameter can represent the voltage applied to mandrel 25 (e.g. potential difference between a nozzle of nozzle assembly 35 and mandrel 25). In some embodiments, the monitored rotating assembly 20 parameter represents the level of alignment of mandrel 25 (e.g. to confirm proper fixation of mandrel 25 in rotating assembly 20, and/or detect undesired wobbling of mandrel 25).

In some embodiments, diagnostic assembly 50 is constructed and arranged to monitor one or more controller 40 parameters, such as to detect an undesired state of the one or more of those parameters. For example, the undesired state correlates to one or more of these controller 40 parameters exceeding a threshold. In some embodiments, the controller 40 monitored parameter can represent a parameter selected from the group consisting of: an input level to power supply 45; an output level of power supply 45; status of an electrical connection within controller 40 and/or another component of system 10; and combinations of one or more of these or other parameters.

In some embodiments, diagnostic assembly 50 is constructed and arranged to monitor one or more tubular conduit parameters (e.g. a tubular conduit 120 described in reference to FIGS. 2, 2A, 2B, or 3 herebelow), such as to detect an undesired state of the one or more of those parameters. For example, the undesired state correlates to one or more of these tubular conduit 120 parameters exceeding a threshold. In some embodiments, the tubular conduit 120 parameter comprises a parameter selected from the group consisting of: diameter; trauma level (e.g. trauma level quantifying a ripped or otherwise disrupted wall of tubular conduit 120); leak in a wall of tubular conduit 120 (e.g. a level of a leak due to an insufficiently ligated sidebranch of a tubular conduit 120 comprising a harvested vein); and combinations of one or more of these or other parameters.

In some embodiments, diagnostic assembly 50 is constructed and arranged to monitor one or more fiber matrix parameters (e.g. a fiber matrix 110 described in reference to FIGS. 2, 2A, 2B, or 3 herebelow), such as to detect an undesired state of the one or more of those parameters. For example, the undesired state correlates to one or more of these fiber matrix 110 parameters exceeding a threshold. In some embodiments, the fiber matrix 110 parameter comprises a parameter selected from the group consisting of: thickness; dryness; fiber diameter; fiber average diameter; fiber diameter range; nodal density; fiber alignment; fiber flatness; fiber twist; fiber elasticity; fiber crystallinity; fiber conformity to target; fiber water content; fiber matrix porosity; fiber matrix thickness; fiber matrix density; fiber matrix thickness distribution along the longitudinal and circumferential axes; and combinations of one or more of these or other parameters.

In some embodiments, graft device 100 comprises one or more spines (e.g. a spine 210 of FIG. 2, 2a, 2b, or 3 herebelow) and diagnostic assembly 50 is constructed and arranged to monitor one or more spine 210 parameters, such as to detect an undesired state of the one or more of those parameters. For example, the undesired state correlates to one or more of these spine 210 parameters exceeding a threshold. In some embodiments, the spine 210 parameter comprises a parameter selected from the group consisting of: position of spine 210 about tubular conduit 120; spine size; spine position; compression level applied to tubular conduit; and combinations of one or more of these or other parameters.

In some embodiments, diagnostic assembly 50 is constructed and arranged to monitor one or more graft device 100 parameters (e.g. a graft device 100 described in reference to FIGS. 2, 2A, 2B, or 3 herebelow), such as to detect an undesired state of the one or more of those parameters. For example, the undesired state correlates to one or more of these graft device 100 parameters exceeding a threshold. In some embodiments, the graft device 100 parameter comprises a parameter selected from the group consisting of: diameter; solvent level; and combinations of one or more of these or other parameters.

In some embodiments, system 10 includes an information element, such as ID 80 shown in FIG. 1. In some embodiments, diagnostic assembly 50 comprises a reader 85 constructed and arranged to read or otherwise extract information from ID 80. ID 80 can comprise an information providing element selected from the group consisting of: barcode; microchip; radio-frequency identification (RFID); and combinations of one or more of these or other elements. Reader 85 can comprise a barcode reader, a microchip reader, and/or an RFID reader. In some embodiments, ID 80 comprises information related to polymer provided to polymer reservoir 33. Reader 85 can extract the information from ID 80 such that diagnostic assembly 50 can determine applicability of the polymer, such as type of polymer used and/or expiration date of the polymer (e.g. detect an undesired state if an improper polymer is being used and/or the polymer expiration date has been surpassed).

In some embodiments, diagnostic assembly 50 comprises a timer used to measure the elapsed time during one or more processes performed by system 10. In these embodiments, if the elapsed time for a process exceeds a threshold, an undesired state is detected (e.g. fiber application time below a minimum or above a maximum time period).

In some embodiments, system 10 is constructed and arranged to correct an undesired state detected by diagnostic assembly 50, such as by modifying one or more parameters of system 10, such as in a closed loop fashion using information provided by diagnostic assembly 50.

In some embodiments, controller 40 comprises an alarm assembly, such as alarm assembly 48 shown, which can be constructed and arranged to be activated when an undesired state is detected, such as to notify an operator of system 10. Alarm assembly 48 can comprise an alarm assembly constructed and arranged to provide an alert selected from the group consisting of: audible alert; visual alert; tactile alert; and combinations of one or more of these alerts. In some embodiments, when an undesired state is detected, application of fiber matrix 110 to tubular conduit 120 is stopped.

Referring now to FIG. 2, a side, partial cut-away view of an example graft device is illustrated. Graft device 100 includes tubular conduit 120 and fiber matrix 110. In some embodiments, graft device 100 further includes spine 210 as shown. Tubular conduit 120 is circumferentially surrounded by fiber matrix 110. Graft device 100 includes a first end 101 and a second end 102, and is preferably configured to be placed between a first body location and a second body location of a patient. Graft device 100 includes lumen 103 from first end 101 to second end 102, such as to carry blood or other fluid when graft device 100 is connected between two body locations, such as between two blood vessels in a cardiovascular bypass procedure.

Tubular conduit 120 can comprise a varying circumferential shape (e.g. an outer surface comprising one or more of: an undulating contour; a tapered contour; one or more bumps, peaks, ridges, divots and/or valleys; and/or a changing cross sectional geometry), and fiber matrix 110 and/or spine 210 can be constructed and arranged to conform to the varying circumferential shape of conduit 120. Conduit 120 can comprise harvested tissue, such as a segment of a harvested vessel, such as a saphenous vein or other vein. In some embodiments, conduit 120 comprises tissue selected from the group consisting of: saphenous vein; vein; artery; urethra; intestine; esophagus; ureter; trachea; bronchi; duct; fallopian tube; and combinations of one or more of these or other tissues. Alternatively or additionally, conduit 120 can comprise artificial material, such as a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations of one or more of these or other materials. In some embodiments, conduit 120 can comprise one or more polymers that are coated (e.g. sputter-coated) with one or more inert materials such as graphite or an inert metal (e.g. gold or platinum), such as to make a surface of conduit 120 conductive.

Fiber matrix 110 can comprise one or more layers, such as a fiber matrix 110 with a thickness between 100 μm and 1000 μm, such as a thickness between 150 μm and 400 μm, between 220 μm and 280 μm, or approximately 250 μm. In some embodiments, fiber matrix 110 comprises an inner layer and an outer layer, such as an inner and outer layer with a spine 210 positioned therebetween, such as is described in reference to FIG. 2B herebelow. Fiber matrix 110 can comprise a matrix of fibers with an average diameter (hereinafter “diameter”) of at least 5 μm, such as a diameter between 6 μm and 15 μm, such as a matrix of fibers with an average diameter of approximately 7.8 μm or approximately 8.6 μm. Fiber matrix 110 can comprise an average porosity (hereinafter “porosity”) of between 40% and 80%, such as a fiber matrix with an average porosity of 50.4% or 46.9%. The porosity of fiber matrix 110 can be selected to control infiltration of materials into fiber matrix 110 and/or to control the rate of transmural cellular infiltration within the fiber matrix 110. In some embodiments, fiber matrix 110 comprises an average compliance (hereinafter “compliance”) between approximately 0.2×10⁻⁴/mmHg and 3.0×10⁻⁴/mmHg when measured in arterial pressure ranges. In some embodiments, fiber matrix 110 comprises an average circumferential elastic modulus (hereinafter “elastic modulus”) between 10 MPa and 18 MPa.

Fiber matrix 110 can comprise at least one polymer such as one or more polymers selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of one or more of these or other polymers. The polymer can be applied in combination with a solvent, such as when the solvent is selected from the group consisting of: hexafluoroisopropanol (HFIP); acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations of one or more of these or other materials. Fiber matrix 110 can comprise a thermoplastic co-polymer including two or more materials, such as a first material and a harder second material. In some embodiments, the softer material comprises segments including polydimethylsiloxane and polyhexamethylene oxide, and the harder material comprises segments including aromatic methylene diphenyl isocyanate. In some embodiments, fiber matrix 110 comprises relatively equal amounts of the softer and harder materials. In some embodiments, fiber matrix 110 comprises Elast-Eon™ material manufactured by Aortech Biomaterials of Scoresby, Australia, such as model number E2-852 with a durometer of 55D.

In some embodiments, fiber matrix 110 is produced by a fiber matrix delivery assembly such as an electrospinning device that converts a polymer solution into fibers applied to tubular conduit 120, such as is described herebelow in reference to system 10 and electrospinning device 400 of FIG. 3. The polymer solution can comprise one or more polymers dissolved in a solvent such as hexafluoroisopropanol (HFIP). In some embodiments, at least a portion of fiber matrix 110 is applied with a device selected from the group consisting of: an electrospinning device; a melt-spinning device; a melt-electrospinning device; a misting assembly; a sprayer; an electrosprayer; a three-dimensional printer; and combinations of one or more of these or other devices.

Fiber matrix 110 can comprise one or more relatively durable (i.e. non-biodegradable) materials and/or one or more biodegradable materials. In some embodiments, fiber matrix 110 comprises a material selected from the group consisting of polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of one or more of these or other materials. In some embodiments, fiber matrix 110 can comprise one or more polymers that are coated (e.g. sputter-coated) with one or more inert materials such as graphite or an inert metal (e.g. gold or platinum), such as to make a surface of conduit 120 conductive. Fiber matrix 110 can comprise one or more drugs or other agents, such as one or more agents constructed and arranged to be released over time.

In some embodiments, graft device 100 further includes one or more kink resisting elements, such as spine 210. Spine 210 can be constructed and arranged to prevent graft device 100 from undergoing undesired motion such as kinking or other narrowing, such as narrowing caused during an implantation procedure and/or while under stresses endured during its functional lifespan. In some embodiments, spine 210 surrounds conduit 120, positioned between conduit 120 and fiber matrix 110. In these embodiments, spine 210 can comprise a diameter approximating the outer diameter (OD) of conduit 120. In some embodiments, spine 210, in whole or in part, can be positioned between one or more layers of fiber matrix 110, such as is shown in FIG. 2B and described herebelow. In some embodiments, spine 210, in whole or in part, can surround the outer surface of fiber matrix 110. In some embodiments, spine 210 is positioned within conduit 120. In some embodiments, multiple spines 210 can be included, each contacting the outer surface of tubular conduit 120, surrounding the outer surface of fiber matrix 110, and/or positioned between two or more layers of fiber matrix 110. In some embodiments, spine 210 comprises two or more spines and/or other kink resisting elements.

Fiber matrix 110 and/or spine 210 can be constructed and arranged to provide one or more functions selected from the group consisting of: minimizing undesirable conditions, such as buckling or kinking, conduit 120 deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through graft device 100, blood flow proximal to graft device 100 and/or blood flow distal to graft device 100; preventing bending and/or allowing proper bending of the graft device 100, such as bending that occurs during and/or after the implantation procedure; preventing accumulation of debris; preventing stress concentration on the tubular wall; maintaining a defined geometry in tubular conduit 120; preventing axial rotation about the length of tubular conduit 120; and combinations of one or more of these or other functions. Spine 210 and fiber matrix 110 can comprise similar elastic moduli, such as to avoid dislocations and/or separations between the two components over time, such as when graft device 100 undergoes cyclic motion and/or strain.

Spine 210 can be applied around conduit 120 prior to, during and/or after application of fiber matrix 110 to graft device 100. For example, spine 210 can be applied prior to application of fiber matrix 110 when spine 210 is positioned between conduit 120 and the inner surface of fiber matrix 110. Spine 210 can be applied during application of fiber matrix 110 when spine 210 is positioned between one or more layers of fiber matrix 110, such as is shown in FIG. 2B. Spine 210 can be applied after application of fiber matrix 110 when spine 210 is positioned outside of fiber matrix 110. Spine 210 can be applied about conduit 120 and/or at least a layer of fiber matrix 110 with one or more tools, such as tool 300 described herebelow in reference to FIG. 3.

Spine 210 can include one or more portions that are resiliently biased, such as a resilient bias configured to provide a radial outward force at locations proximate ends 101 and/or 102, such as to provide a radial outward force to support or enhance the creation of an anastomosis during a cardiovascular bypass procedure. In some embodiments, spine 210 includes one or more portions that are malleable.

Spine 210 can include multiple curved projections 211′ and 211″, collectively 211. Projections 211′ each include a tip portion 212′ and projections 211″ each include a tip portion 212″ (collectively, tip portions 212). Tip portions 212 can be arranged in the overlapping arrangement shown in FIG. 2. Projections 211′ and 211″ can comprise a first and second support portion, respectively, that are arranged such that at least one rotates relative to the other to create an opening to receive tubular conduit 120. In some embodiments, each tip portion 212 can comprise a diameter between 0.020 inches and 0.064 inches, such as a diameter approximating 0.042 inches. Projections 211 can each comprise a loop of a filament (e.g. a loop of a continuous filament), and projections 211′ and 211″ can be arranged in an interdigitating arrangement such as the alternating, interdigitating arrangement shown in FIG. 2. In some embodiments, the interdigitating projections 211′ and 211″ can overlap (e.g. spine 210 covers more than 360° of conduit 120). In some embodiments, projections 211′ and 211″ are arranged with an overlap of at least 1.0 mm, at least 1.1 mm or at least 1.4 mm. In some embodiments, spine 210 can be constructed and arranged as described in applicant's co-pending International Patent Application Ser. No. PCT/US2014/056371, filed Sep. 18, 2014, the contents of which are incorporated herein by reference in their entirety.

Spine 210 can comprise at least three projections 211, such as at least six projections 211. In some embodiments, spine 210 includes at least two projections 211 for every 15 mm of length of spine 210, such as at least two projections 211 for every 7.5 mm of length of spine 210, or at least two projections for every 2 mm of length of spine 210. In some embodiments, spine 210 comprises two projections 211 for each approximately 6.5 mm of length of spine 210. In some embodiments, a series of projections 211 are positioned approximately 0.125 inches from each other.

Spine 210 can comprise one or more continuous filaments 216, such as three or less continuous filaments, two or less continuous filaments, or a single continuous filament. In some embodiments, spine 210 comprises a continuous filament 216 of at least 15 inches long (i.e. the curvilinear length), or at least 30 inches long, such as when spine 210 comprises a length of approximately 3.5 inches. In some embodiments, filament 216 comprises a length (e.g. a continuous curvilinear length or a sum of segments with a cumulative curvilinear length) of approximately 65 inches (e.g. to create a 4.0 mm diameter spine 210), or a length of approximately 75 inches (e.g. to create a 4.7 mm diameter spine 210), or a length of approximately 85 inches (e.g. to create a 5.5 mm diameter and/or 3.5 inches long spine 210). Filament 216 can comprise a relatively continuous cross section, such as an extruded or molded filament with a relatively continuous cross section. Spine 210 can comprise a filament 216 including at least a portion with a cross sectional geometry selected from the group consisting of: elliptical; circular; oval; square; rectangular; trapezoidal; parallelogram-shaped; rhomboid-shaped; T-shaped; star-shaped; spiral-shaped; (e.g. a filament comprising a rolled sheet); and combinations of one or more of these or other geometries. Filament 216 can comprise a cross section with a major axis between approximately 0.2 mm and 1.5 mm in length, such as a circle or oval with a major axis less than or equal to 1.5 mm, less than or equal to 0.8 mm, or less than or equal to 0.6 mm, or between 0.4 mm and 0.5 mm. Filament 216 can comprise a cross section with a major axis greater than or equal to 0.1 mm, such as a major axis greater than or equal to 0.3 mm. In some embodiments, the major axis and/or cross sectional area of filament 216 is proportionally based to the diameter of spine 210 (e.g. a larger spine 210 diameter correlates to a larger filament 216 diameter, such as when a range of different diameter spine 210's are provided in a kit as described herebelow in reference to FIG. 3.

Filament 216 can be a single core, monofilament structure. Alternatively, filament 216 can comprise multiple filaments, such as a braided multiple filament structure. In some embodiments, filament 216 can comprise an injection molded component or a thermoset plastic component, such as when spine 210 comprises multiple projections 211 that are created at the same time as the creation of one or more filaments 216 (e.g. when filament 216 is created in a three dimensional biased shape).

Filament 216 can comprise an electrospun component, such as a component produced by the same electrospinning device used to create fiber matrix 110, such as when spine 210 and fiber matrix 110 comprise the same or similar materials.

Spine 210 can comprise a material with a durometer between 52 D and 120 R, such as between 52 D and 85 D, such as between 52 D and 62 D. In some embodiments, spine 210 comprises a material with a durometer of approximately 55 D. Spine 210 can comprise one or more polymers, such as a polymer selected from the group consisting of: silicone; polyether block amide; polypropylene; nylon; polytetrafluoroethylene; polyethylene; ultra high molecular weight polyethylene; polycarbonates; polyolefins; polyurethanes; polyvinylchlorides; polyamides; polyimides; polyacrylates; polyphenolics; polystyrene; polycaprolactone; polylactic acid; polyglycolic acid; polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of one or more of these or other materials.

Spine 210 can comprise the same or substantially similar material(s) as fiber matrix 110. Spine 210 can comprise at least one thermoplastic co-polymer. Spine 210 can comprise two or more materials, such as a first material and a second material harder than the first material. In some embodiments, Spine 210 comprises relatively equal amounts of a harder material and a softer material. The softer material can comprise polydimethylsiloxane and a polyether-based polyurethane, and the harder material can comprise aromatic methylene diphenyl isocyanate. Spine 210 can comprise one or more drugs or other agents, such as one or more agents constructed and arranged to be released over time.

In some embodiments, spine 210 comprises a metal material, such as a metal selected from the group consisting of: nickel titanium alloy; titanium alloy; titanium; stainless steel; tantalum; magnesium; cobalt-chromium alloy; gold; platinum; and combinations of one or more of these or other materials. In some embodiments, spine 210 comprises a reinforced resin, such as a resin reinforced with carbon fiber and/or Kevlar. In some embodiments, at least a portion of spine 210 is biodegradable, such as when spine 210 comprises a biodegradable material such as a biodegradable metal or biodegradable polymer. In these embodiments, fiber matrix 110 can further comprise a non-biodegradable material. In some embodiments, spine 210 does not comprise a biodegradable material.

Spine 210 can be configured to biodegrade over time such as to provide a temporary kink resistance or other function to graft device 100. In one embodiment, spine 210 can temporarily provide kink resistance to graft device 100 for a period of less than twenty-four hours. In an alternative embodiment, spine 210 can provide kink resistance to graft device 100 for a period of less than one month. In yet another embodiment, spine 210 can provide kink resistance to graft device 100 for a period of less than six months. Numerous forms of biodegradable materials can be employed. Bolz et al. (U.S. Pat. No. 6,287,332) discloses a bioabsorbable implant which includes a combination of metal materials that can be an alloy or a local galvanic element. Metal alloys can consist of at least a first component which forms a protecting passivation coat and a second component configured to ensure sufficient corrosion of the alloy. The first component is at least one component selected from the group consisting of: magnesium, titanium, zirconium, niobium, tantalum, zinc and silicon, and the second component is at least one metal selected from the group consisting of: lithium, sodium, potassium, manganese, calcium and iron. Furst et al. (U.S. patent application Ser. No. 11/368,298) discloses an implantable device at least partially formed of a bioabsorbable metal alloy that includes a majority weight percent of magnesium and at least one metal selected from calcium, a rare earth metal, yttrium, zinc and/or zirconium. Doty et al. (U.S. patent application Ser. No. 11/744,977) discloses a bioabsorbable magnesium reinforced polymer stent that includes magnesium or magnesium alloys. Numerous biodegradable polymers can be used such as are described hereabove.

Fiber matrix 110 and/or spine 210 can comprise one or more coatings. The one or more coatings can comprise an adhesive element or otherwise exhibit adhesive properties, such as a coating comprising a material selected from the group consisting of: fibrin gel; a starch-based compound; mussel adhesive protein; and combinations of one or more of these or other materials. The coating can be constructed and arranged to provide a function selected from the group consisting of: anti-thrombogenecity; anti-proliferation; anti-calcification; vasorelaxation; and combinations of one or more of these or other functions. A coating can comprise a dehydrated gelatin, such as a dehydrated gelatin coating configured to hydrate to cause adherence of two or more of tubular conduit 120, fiber matrix 110 and spine 210. A coating can comprise a hydrophilic and/or a hydrophobic coating. A coating can comprise a radiopaque coating. In some embodiments, spine 210 comprises at least a portion that is radiopaque, such as when spine 210 comprises a radiopaque material such as barium sulfate.

In some embodiments, graft device 100 is constructed and arranged to be placed in an in-vivo geometry including one or more arced portions including a radius of curvature of as low as 0.5 cm (e.g. without kinking). In some embodiments, graft device 100 is produced using system 10 and/or electrospinning device 400 of FIG. 3, as described herebelow.

Referring now to FIG. 2A, a sectional view of an example embodiment of the graft device of FIG. 2 is illustrated, comprising a tubular conduit and a surrounding fiber matrix. Graft device 100 includes tubular conduit 120. A fiber matrix 110 has been applied about the surface of conduit 120, such as is described in detail herebelow in reference to FIG. 3. Fiber matrix 110 can comprise one or more polymers, such as a combination of polydimethylsiloxane and polyhexamethylene oxide soft segments, and aromatic methylene diphenyl isocyanate hard segments. Fiber matrix 110 can comprise a thickness of between 220 μm and 280 μm, such as a thickness of approximately 250 μm.

Referring now to FIG. 2B, a sectional view of another example embodiment of the graft device of FIG. 2 is illustrated, including a spine placed between layers of a fiber matrix. In the embodiment of FIG. 2B, spine 210 has been placed between one or more inner layers of fiber, inner layer 110a, and one or more outer layers of fiber, outer layer 110b. In these embodiments, spine 210 can be applied (e.g. laterally applied) to conduit 120 after inner layer 110a has been applied to conduit 120. Spine 210 can be applied by an electrospinning device or other fiber matrix delivery assembly, as described herein, such as by interrupting the delivery of fiber to conduit 120, to apply spine 210 over the already applied inner layer 110a. In some embodiments, inner layer 110a comprises a thickness approximately one-half the thickness of outer layer 110b. In some embodiments, inner layer 110a comprises a thickness of approximately between 62 μm and 83 μm. In some embodiments, inner layer 110a comprises between 1% and 99% of the total thickness of fiber matrix 110, such as between 25% and 60% of the total thickness, or approximately 33% of the total thickness of fiber matrix 110. In some embodiments, the process time of applying inner layer 110a is between 1% and 99% of the total application time (i.e. the collective time to apply inner layer 110a and outer layer 110b), such as between 25% and 60% of the total fiber application time, or approximately 33% of the total fiber application time.

Spine 210 comprises an inner surface 218 which contacts the outer surface of inner layer 110a. Spine 210 further comprises an outer surface 219 which contacts the inner surface of outer layer 110b. Inner surface 218, outer surface 219 and/or another surface of spine 210 (e.g. one or more surfaces between inner surface 218 and outer surface 219) can comprise a coating, such as a coating described hereabove.

Application of layers 110a and 110b can be performed as is described in detail herebelow in reference to FIG. 3. Fiber matrix layers 110a and/or 110b can comprise one or more polymers, such as a combination of polydimethylsiloxane and polyhexamethylene oxide soft segments, and aromatic methylene diphenyl isocyanate hard segments. Layers 110a and/or 110b can comprise a matrix of fibers with a diameter between 6 μm and 15 μm, such as a matrix of fibers with an average diameter of approximately 7.8 μm or approximately 8.6 μm. Layers 110a and/or 110b can comprise a porosity of between 40% and 80%, such as a fiber matrix with an average porosity of 50.4% or 46.9%. In some embodiments, layers 110a and/or 110b comprise a compliance between approximately 0.2×10⁻⁴/mmHg and 3.0×10⁻⁴/mmHg when measured in arterial pressure ranges. In some embodiments, fiber matrix 110 comprises an elastic modulus between 10 MPa and 18 MPa.

Referring now to FIG. 3, a schematic view of an example system for producing a graft device with an electrospun fiber matrix is illustrated. System 10 includes a fiber matrix delivery assembly, electrospinning device 400. System 10 is constructed and arranged to produce one or more graft devices, such as graft device 100′ or 100″ shown (singly or collectively graft device 100), each including a fiber matrix, such as fiber matrix 110′ or 110″, respectively (singly or collectively fiber matrix 110). Fiber matrix 110′ and 110″ each surround a tubular conduit, tubular conduits 120′ and 120″, respectively. Electrospinning device 400 includes diagnostic assembly 50. Diagnostic assembly 50 is constructed and arranged to detect an undesired state of at least one of system 10 and/or graft device 100. Diagnostic assembly 50 can be constructed and arranged similar to diagnostic assembly 50 of FIG. 1, described hereabove. System 10 can include one or more sensors, such as one or more sensors 11, also described hereabove in reference to FIG. 1. Diagnostic assembly 50 can process signals received from sensors 11 or other components of system 10, to determine if an undesired state has been detected.

System 10 includes rotating assembly 20 which includes mandrel 250, about which a tubular conduit 120 has been placed. System 10 can include polymer material 111, including a mixture of one or more polymers, solvents and/or other materials used to create fiber matrix 110, such as are described hereabove in reference to FIG. 2. Tubular conduit 120 can include living tissue and/or artificial materials. In some embodiments, system 10 comprises one or more similar or dissimilar spines 210, and graft device 100 comprises one or more of the spines 210. System 10 can include spine application tool 300, which can comprise a manual or automated (e.g. robotic) tool used to place spine 210 about tubular conduit 120, such as between one or more layers of fiber matrix 110 (e.g. between an inner layer with a first thickness, and an outer layer with a second thickness approximately twice as thick as the first layer's thickness). In some embodiments, graft device 100, fiber matrix 110, spine 210, and/or conduit 120 are constructed and arranged as is described hereabove in reference to FIG. 2. In some embodiments, system 10 can include one or more tools, components, assemblies and/or otherwise be constructed and arranged as described in applicant's co-pending International Patent Application Ser. No. PCT/US2014/056371, filed Sep. 18, 2014, the contents of which is incorporated herein by reference in their entirety.

Mandrel 250 can comprise a metal mandrel, such as a mandrel constructed of 304 or 316 series stainless steel. Mandrel 250 can comprise a mirror-like surface finish, such as a surface finish with an Rₐ of approximately 0.1 μm to 0.8 μm. Mandrel 250 can comprise a length of up to 45 cm, such as a length of between 30 cm and 45 cm, or between 38 cm and 40 cm. In some embodiments, system 10 includes multiple mandrels 250 with multiple different geometries, such as a set of mandrels 250 with different diameters (e.g. diameters of 3.0 mm, 3.5 mm, 4.0 mm, and/or 4.5 mm). Each end of mandrel 250 is inserted into driving elements of rotating assembly 20, motors 440a and 440b, respectively, such that mandrel 250 can be rotated about axis 435 during application of fiber matrix 110. In some embodiments, a single motor drives one end of mandrel 250, with the opposite end attached to a rotatable attachment element (e.g. a bearing) of electrospinning device 400.

Electrospinning device 400 can include one or more polymer delivery assemblies, and in the illustrated embodiment, device 400 includes polymer delivery assembly 30. Polymer delivery assembly 30 comprises nozzle assembly 405, which can be constructed and arranged similar to nozzle assembly 35 of FIG. 1. Nozzle assembly 405 includes nozzle 427 including an orifice constructed and arranged to deliver fiber matrix 110 to tubular conduit 120. Nozzle 427 can be a tubular structure including nozzle central axis 428. Nozzle assembly 405 is fluidly attached to polymer solution dispenser 401 via delivery tube 425. Polymer solution dispenser 401 can be constructed and arranged similar to polymer reservoir 33 of FIG. 1. Dispenser 401 can comprise material supplied by polymer material 111 (e.g. when polymer material 111 comprises one or more polymers contained in a cartridge that is operably received by polymer solution dispenser 401). Polymer delivery assembly 30 further comprises linear drive assembly 445. Nozzle assembly 405 is operably attached to linear drive assembly 445, which is configured to translate nozzle assembly 405 in at least one direction for a linear travel distance D_{SWEEP} as shown. In some embodiments, D_{SWEEP} comprises a length of approximately 30 cm, such as a length of at least 10 cm, 20 cm, 30 cm, 35 cm, or 40 cm.

In some embodiments, polymer material 111 comprises two or more polymers, such as a first polymer with a first hardness, and a second polymer with a second hardness different than the first hardness. Polymer material can comprise a mixture of similar or dissimilar amounts of polyhexamethylene oxide soft segments, and aromatic methylene diphenyl isocyanate hard segments. Polymer material 111 can further comprise one or more solvents, such as HFIP (e.g. HFIP with a 99.97% minimum purity). Polymer material 111 can comprise one or more polymers in a concentrated solution fully or at least partially solubilized within a solvent and comprise a polymer weight to solvent volume ratio between 20% and 35%, a typical concentration is between 24% and 26% (more specifically between 24.5% and 25.5%). Polymer material 111 can comprise one or more materials with a molecular weight average (M_{w}) between 80,000 and 150,000 (PDI−M_{w}/Mₙ=2.1−3.5). Polymer material 111 can comprise a polymer solution with a viscosity between 2000 cP and 2400 cP (measured at 25° C. and with shear rate=20s⁻¹). Polymer material 111 can comprise a polymer solution with a conductivity between 0.4 μS/cm and 1.7 μS/cm (measured at a temperature between 20° C. and 22° C.). Polymer material 111 can comprise a polymer solution with a surface tension between 21.5 mN/m and 23.0 mN/m (measured at 25° C.). In some embodiments, system 10 is constructed and arranged to produce a fiber matrix 110 with a thickness (absent of any spine 210) of between approximately 220 μm and 280 μm. Fiber matrix 110 can comprise a matrix of fibers with a diameter between 6 μm and 15 μm, such as a matrix of fibers with an average diameter of approximately 7.8 μm or approximately 8.6 μm. Fiber matrix 110 can comprise a porosity of between 40% and 80%, such as a fiber matrix with an average porosity of 50.4% or 46.9%. In some embodiments, fiber matrix 110 comprises a compliance between approximately 0.2×10⁻⁴/mmHg and 3.0×10⁻⁴/mmHg when measured in arterial pressure ranges. In some embodiments, fiber matrix 110 comprises an elastic modulus between 10 MPa and 18 MPa.

Nozzle assembly 405 can be configured to deliver polymer material 111 to nozzle 427 at a flow rate of between 10 ml/hr and 25 ml/hr, such as at a flow rate of approximately 15 ml/hr or 20 ml/hr.

As described above, in some embodiments, system 10 is constructed and arranged to produce a graft device 100 including a spine 210. Spine 210 can comprise multiple spines 210 with different inner diameters (IDs), such as multiple spines with IDs of approximately 4.0 mm, 4.7 mm, and/or 5.5 mm. Spine 210 can comprise a filament with a diameter of approximately 0.4 mm (e.g. for a spine with an ID between 4.0 mm and 4.7 mm). Spine 210 can comprise a filament with a diameter of approximately 0.5 mm (e.g. for a spine with an ID between 4.8 mm and 5.5 mm). Spine 210 can comprise a series of inter-digitating fingers spaced approximately 0.125 inches from each other so that the recurring unit of spine including one left finger and one right finger occurs every 0.25 inches. This recurring feature length can have a range comprised between 0.125 inches and 0.375 inches. The fingers can overlap in a symmetric or asymmetric pattern, such as an overlap of opposing fingers between 2.5 mm and 1.0 mm around the circumferential perimeter of spine 210. Spine 210 can be heat treated to achieve a resilient bias. Spine 210 can be surface-treated (e.g. with dimethylformamide) to increase the surface roughness and reduce crystallinity (e.g. to improve solvent-based adhesion with the deposited electrospun material, fiber matrix 110).

System 10 can include drying assembly 310, which is constructed and arranged to remove moisture from tubular conduit 120. In some embodiments, tubular conduit 120 comprises harvested tissue (e.g. a harvested saphenous vein segment) and drying assembly 310 comprises gauze or other material used to manually remove fluids from tubular conduit 120, such as to improve adherence between fiber matrix 110 and tubular conduit 120.

Electrospinning device 400 can include one or more graft modification assemblies constructed and arranged to modify one or more components and/or one or more portions of graft device 100. In the illustrated embodiment, device 400 includes modification assembly 70. Modification assembly 70 can be constructed and arranged similar to modification assembly 70 of FIG. 1. Modification assembly 70 comprises a nozzle assembly or other modifying assembly 605, which includes modifying element 627. Modification assembly 70 further comprises linear drive assembly 645. Assembly 605 is operably attached to linear drive assembly 645, which is configured to translate assembly 605 in at least one direction, such as a reciprocating motion in back and forth directions spanning a distance similar to D_{SWEEP} of linear drive assembly 445. Assembly 605 can be operably attached to supply 620 via delivery tube 625. System 10 can include one or more graft device 100 modifying agents, such as agent 502. Agent 502 can comprise a solvent configured to perform a surface modification, such as a solvent selected from the group consisting of: dimethylformamide; hexafluoroisopropanol; tetrahydrofuran; dimethyl sulfoxide; isopropyl alcohol; ethanol; and combinations of one or more of these or other solvents. In some embodiments, system 10 is constructed and arranged to perform a surface modification configured to enhance the adhesion of two or more of tubular conduit 120, spine 210 and fiber matrix 110. In some embodiments, system 10 is constructed and arranged to perform a surface modification to fiber matrix 110 and/or spine 210 to cause a modification of the surface energy of fiber matrix 110 and/or spine 210, respectively. In some embodiments, the surface of spine 210 is modified with a heated die comprising a textured or otherwise non-uniform surface. In some embodiments, electrospinning device 400 and/or another component of system 10 comprise a radiofrequency plasma glow discharge assembly constructed and arranged to perform a surface modification of spine 210, such as a process performed in the presence of a material selected from the group consisting of: hydrogen; nitrogen; ammonia; oxygen; carbon dioxide; C₂F₆; C₂F₄; C₃F₆; C₂H₄; CH₄; and combinations of one or more of these or other materials.

Supply 620 can comprise one or more of: a reservoir of one or more agents, such as agent 502; a power supply such as a laser power supply; and a reservoir of compressed fluid. In some embodiments, modifying element 627 comprises a nozzle, such as a nozzle configured to deliver a fiber matrix 110 modifying agent, tubular conduit 120 modifying agent, spine 210 modifying agent, and/or a graft device 100 modifying agent. For clarification, any reference to a “nozzle” or “assembly”, in singular or plural form, can include one or more nozzles, such as one or more nozzles 427, or one or more assemblies, such as one or more nozzle assemblies 405 or one or more modifying assemblies 605.

In some embodiments, modifying element 627 is configured to deliver an agent 502 comprising a wax or other protective substance to tubular conduit 120 prior to the application of fiber matrix 110, such as to prevent or otherwise minimize exposure of tubular conduit 120 to one or more solvents (e.g. HFIP) included in polymer material 111.

In some embodiments, modifying element 627 is configured to deliver a kink resisting element, for example spine 210, such as a robotic assembly constructed and arranged to laterally deliver spine 210 about at least conduit 120 (e.g. about conduit 120 and an inner layer of fiber matrix 110). Alternatively or additionally, modifying element 627 can be configured to modify conduit 120, spine 210 and/or fiber matrix 110, such as to cause graft device 100 to be kink resistant or otherwise enhance the performance of the graft device 100 produced by system 10. In these graft device 100 modifying embodiments, modifying element 627 can comprise a component selected from the group consisting of: a robotic device such as a robotic device configured to apply spine 210 to tubular conduit 120; a nozzle, such as a nozzle configured to deliver agent 502; an energy delivery element such as a laser delivery element such as a laser excimer diode or CO₂ laser, or another element configured to trim one or more components of graft device 100; a fluid jet such as a water jet or air jet configured to deliver fluid during the application of fiber matrix 110 to conduit 120; a cutting element such as a cutting element configured to trim spine 210 and/or fiber matrix 110; a mechanical abrader; and combinations of one or more of these or other components. Modification of fiber matrix 110 or other graft device 100 component by modifying element 627 can occur during the application of fiber matrix 110 and/or after fiber matrix 110 has been applied to conduit 120. Modification of one or more spines 210 can be performed prior to and/or after spine 210 has been applied to surround conduit 120. In some embodiments, modifying element 627 can be used to cut or otherwise trim fiber matrix 110 and/or a spine 210.

In an alternative embodiment, modification assembly 70 of system 10 can be an additional component or assembly, separate from electrospinning device 400, such as a handheld device configured to deliver spine 210. In some embodiments, modification assembly 70 comprises a handheld laser, such as a laser device which can be hand operated by an operator. Modification assembly 70 can be used to modify graft device 100 after removal from electrospinning device 400, such as prior to and/or during an implantation procedure.

Laser or other modifications to fiber matrix 110 can cause portions of fiber matrix 110 to undergo physical changes, such as hardening, softening, melting, stiffening, creating a resilient bias, expanding, and/or contracting, and/or can also cause fiber matrix 110 to undergo chemical changes, such as forming chemical bonds with an adhesive layer between the outer surface of conduit 120 and fiber matrix 110. In some embodiments, modifying element 627 is alternatively or additionally configured to modify tubular conduit 120, such that tubular conduit 120 comprises a kink resisting or other performance enhancing element. Modifications to tubular conduit 120 can include but are not limited to a physical change to one or more portions of tubular conduit 120 selected from the group consisting of: drying; hardening; softening; melting; stiffening; creating a resilient bias; expanding; contracting; and combinations of one or more of these or other changes. Modifications of tubular conduit 120 can cause tubular conduit 120 to undergo chemical changes, such as forming chemical bonds with an adhesive layer between an outer surface of conduit 120 and spine 210 and/or fiber matrix 110.

As described herein, fiber matrix 110 can include an inner layer and an outer layer, where the inner layer can include an adhesive component and/or exhibit adhesive properties. The inner layer can be delivered separate from the outer layer, for example, delivered from a separate nozzle or at a separate time during the process. Selective adhesion between the inner and outer layers can be configured to provide kink resistance. Spine 210 can be placed between the inner and outer layers of fiber matrix 110, such as is described hereabove in reference to FIG. 2B.

In some embodiments, electrospinning device 400 can be configured to deliver fiber matrix 110 and/or an adhesive layer according to set parameters configured to produce a kink resistant element in and/or provide kink resisting properties to graft device 100. For example, an adhesive layer can be delivered to conduit 120 for a particular length of time, followed by delivery of a polymer solution for another particular length of time. Other typical application parameters include but are not limited to: amount of adhesive layer and/or polymer solution delivered; rate of adhesive layer and/or polymer solution delivered; nozzle 427 distance to mandrel 250 and/or conduit 120; linear travel distance of nozzle 427 or a fiber modifying element along its respective drive assembly (for example, drive assembly 445 or 645); linear travel speed of nozzle 427 or a fiber modifying element along its respective drive assembly; compositions of the polymer solution and/or adhesive layer; concentrations of the polymer solution and/or adhesive layer; solvent compositions and/or concentrations; fiber matrix 110 inner and outer layer compositions and/or concentrations; spontaneous or sequential delivery of the polymer solution and the adhesive layer; voltage applied to the nozzle; voltage applied to the mandrel; viscosity of the polymer solution; temperature of the treatment environment; relative humidity of the treatment environment; airflow within the treatment environment; and combinations of one or more of these or other parameters.

Nozzle 427 can be constructed of stainless steel, such as passivated 304 stainless steel. In some embodiments, nozzle 427 and nozzle assembly 405 are constructed and arranged as described herebelow in reference to FIG. 4. A volume of space surrounding nozzle 427 can be maintained free of objects or substances which can interfere with the electrospinning process, also as described herebelow in reference to FIG. 4. Nozzle geometry and orientation, as well as the electrical potential voltages applied between nozzle 427 and mandrel 250 are chosen to control fiber generation, such as to create a fiber matrix 110 as described in reference to FIG. 2 hereabove.

Mandrel 250 is positioned in a particular spaced relationship from nozzle assembly 405 and/or assembly 605, and nozzle 427 and/or modifying element 627, respectively. In the illustrated embodiment, mandrel 250 is positioned above and below assemblies 605 and 405, respectively. Alternatively, mandrel 250 can be positioned either above, below, to the right and/or or to the left of, assembly 405 and/or assembly 605. The distance between mandrel 250 and the tip of nozzle 427 and/or modifying element 627 can be less than 20 cm, or less than 15 cm, such as distance of between 12.2 cm and 12.8 cm or approximately 12.5 cm. In some embodiments, multiple nozzles 427 and/or multiple modifying elements 627, for example components of similar or dissimilar configurations, can be positioned in various orientations relative to mandrel 250. In some embodiments, the distance between nozzles 427 and/or modifying elements 627 and mandrel 250 varies along the length of their respective travel along mandrel 250, such as to create a varying pattern of fiber matrix 110 along conduit 120. In some embodiments, nozzle 427 and/or modifying element 627 distances from mandrel 250 can vary continuously during the electrospinning process and/or the distance can vary for one or more set periods of time during the process.

In some embodiments, an electrical potential is applied between nozzle 427 and one or both of conduit 120 and mandrel 250. The electrical potential can draw at least one fiber from nozzle assembly 405 to conduit 120. Conduit 120 can act as the substrate for the electrospinning process, collecting the fibers that are drawn from nozzle assembly 405 by the electrical potential.

In some embodiments, mandrel 250 and/or conduit 120 has a lower voltage than nozzle 427 to create the desired electrical potential. For example, the voltage of mandrel 250 and/or conduit 120 can be a negative or zero voltage while the voltage of nozzle 427 can be a positive voltage. Mandrel 250 and/or conduit 120 can have a voltage of about −5 kV (e.g., −10 kV, −9 kV, −8 kV, −7 kV, −6 kV, −5 kV, −4.5 kV, −4 kV, −3.5 kV, −3.0 kV, −2.5 kV, −2 kV, −1.5 kV, or −1 kV) and the nozzle 427 can have a voltage of about 30 15 kV (e.g., 2.5 kV, 5 kV, 7.5 kV, 12 kV, 13.5 kV, 15 kV, 17 kV, or 20 kV). In some embodiments, the potential difference between nozzle 427 and mandrel 250 and/or conduit 120 can be from about 5 kV to about 30 kV. This potential difference draws fibers from nozzle 427 to conduit 120. In some embodiments, nozzle 427 is electrically charged with a potential of between +15 kV and +17 kV while mandrel 250 is at a potential of approximately −2 kV. In some embodiments, mandrel 250 is a fluid mandrel, such as the fluid mandrel described in applicant's co-pending U.S. patent application Ser. No. 13/997,933, filed Jun. 25, 2013, which is incorporated herein by reference in their entirety.

In some embodiments, system 10 comprises a polymer solution, such as polymer material 111. Polymer material 111 can be introduced into polymer solution dispenser 401, and then delivered to nozzle assembly 405 through polymer solution delivery tube 425. The electrical potential between nozzle 427 and conduit 120 and/or mandrel 250 can draw the polymer solution through nozzle 427 of nozzle assembly 405. Electrostatic repulsion, caused by the fluid becoming charged from the electrical potential, counteracts the surface tension of a stream of the polymer solution at nozzle 427 of the nozzle assembly 405. After the stream of polymer solution is stretched to its critical point, one or more streams of polymer solution emerges from nozzle 427 of nozzle assembly 405, and/or at a location below nozzle assembly 405, and move toward the negatively charged conduit 120. Using a volatile solvent, the solution dries substantially during transit and fiber is applied about conduit 120 creating fiber matrix 110.

Mandrel 250 is configured to rotate about an axis, such as central axis 435 of mandrel 250, with axis 428 of nozzle 427 typically oriented orthogonal to axis 435. In some embodiments, axis 428 of nozzle 427 is horizontally offset from axis 435, such as is described herebelow in reference to FIG. 4. The rotation around axis 435 allows fiber matrix 110 to be applied along all sides, or around the entire circumference of conduit 120. In some embodiments, two motors 440a and 440b are used to rotate mandrel 250. Alternatively, electrospinning device 400 can include a single motor configured to rotate mandrel 250, such as is described hereabove. The rate of rotation of mandrel 250 can determine how the electrospun fibers are applied to one or more segments of conduit 120. For example, for a thicker portion of fiber matrix 110, the rotation rate can be slower than when a thinner portion of fiber matrix 110 is desired. In some embodiments, mandrel 250 is rotated at a rate (e.g. a minimum, maximum or average rate) of between 100 rpm and 400 rpm, such as a rate of between 200 rpm and 300 rpm, between 240 rpm and 260 rpm, or approximately 250 rpm.

In addition to mandrel 250 rotating around axis 435, the nozzle assembly 405 can move, such as when driven by drive assembly 445 in a reciprocating or oscillating horizontal motion (to the left and right of the page). Drive assembly 445, as well as drive assembly 645 which operably attaches to assembly 605, can each comprise a linear drive assembly, such as a belt-driven and/or gear-driven drive assembly comprising two or more pulleys driven by one or more stepper motors. Additionally or alternatively, assemblies 405 and/or 605 can be constructed and arranged to rotate around axis 435, rotating means not shown. The length of drive assemblies 445 and/or 645 and the linear motion applied to assemblies 405 and 605, respectively, can vary based on the length of conduit 120 to which a fiber matrix 110 is delivered and/or a fiber matrix 110 modification is applied. For example, the supported linear motion of drive assemblies 445 and/or 645 can be from about 10 cm to about 50 cm, such as to cause a translation of assembly 405 and/or 605 between 27 cm and 31 cm, or approximately 29 cm. Rotational speeds of mandrel 250 and translational speeds of assemblies 405 and/or 605 can be relatively constant, or can be varied during the fiber application process. In some embodiments, assembly 405 and/or 605 are translated (e.g. back and forth) at a relatively constant translation rate between 40 mm/sec and 150 mm/sec, such as to cause nozzle 427 and/or modifying element 627 to translate at a rate of between 50 mm/sec and 80 mm/sec, between 55 mm/sec and 65 mm/sec, or approximately 60 mm/sec, during the majority of its travel. In some embodiments, system 10 is constructed and arranged to rapidly change directions of translation (e.g. by maximizing deceleration before a direction change and/or maximizing acceleration after a direction change).

Assemblies 405 and/or 605 can move along the entire length and/or along specific portions of the length of conduit 120. In some embodiments, fiber and/or a modification is applied to the entire length of conduit 120 plus an additional 5 cm (to mandrel 250) on either or both ends of conduit 120. In another embodiment, fiber(s) and/or a modification is applied to the entire length of conduit 120 plus at least 1 cm beyond either or both ends of conduit 120. Assemblies 405 and/or 605 can be controlled such that specific portions along the length of conduit 120 are reinforced with a greater amount (e.g. thicker segment) of fiber matrix 110 as compared to other or remaining portions. Alternatively or additionally, assemblies 405 and/or 605 can be controlled such that specific portions of the length of conduit 120 include one or more kink resistant elements (e.g. one or more spines 210) positioned at those one or more specific conduit 120 portions. In addition, conduit 120 can be rotating around axis 435 while assemblies 405 and/or 605 move, via drive assemblies 445 and/or 645, respectively, to position assemblies 405 and/or 605 at the particular portion of conduit 120 to which fiber is applied and/or modified.

System 10 can also include a power supply, power supply 410 configured to provide the electric potentials to nozzle 427 and mandrel 250, as well as to supply power to other components of system 10 such as drive assemblies 445 and 645 and assembly 605. Power supply 410 can be connected, either directly or indirectly, to at least one of mandrel 250 or conduit 120. Power can be transferred from power supply 410 to each component by, for example, one or more wires.

System 10 can include an environmental control assembly including environmental chamber 60 that surrounds electrospinning device 400. System 10 can be constructed and arranged to control the environmental conditions within chamber 60, such as to control one or more areas surrounding nozzle assembly 405 and/or mandrel 250 during the application of fiber matrix 110 to conduit 120. Chamber 60 can include inlet port assembly 61 and outlet port assembly 62. Inlet port assembly 61 and/or outlet port assembly 62 can each include one or more components such as one or more components selected from the group consisting of: a fan; a source of a gas such as a dry compressed air source; a source of gas at a negative pressure; a vapor source such as a source including a buffered vapor, an alkaline vapor and/or an acidic vapor; a filter such as a HEPA filter; a dehumidifier; a humidifier; a heater; a chiller; and electrostatic discharge reducing ion generator; and combinations of these. Chamber 60 can include one or more environmental control components to monitor and/or control temperature, humidity and/or pressure within chamber 60. Chamber 60 can be constructed and arranged to provide relatively uniform ventilation about mandrel 250 (e.g. about tubular conduit 120, fiber matrix 110 and/or spine 210) including an ultra-dry (e.g. ≦2 ppm water or other liquid content) compressed gas (e.g. air) source configured to reduce humidity within chamber 60. Inlet port 61 and outlet port 62 can be oriented to purge air from the top of chamber 60 to the bottom of chamber 60 (e.g. to remove vapors of one or more solvents (e.g. HFIP) which can tend to settle at the bottom of chamber 60). Chamber 60 can be constructed and arranged to replace the internal volume of chamber 60 at least once every 3 minutes, or once every 1 minute, or once every 30 seconds. Outlet port 62 can include one or more filters (e.g. replaceable cartridge filters) which are suitable for retaining halogenated solvents or other undesired materials evacuated from chamber 60. Chamber 60 can be constructed and arranged to maintain a flow rate through chamber 60 of at least 30 L/min, such as at least 45 L/min or at least 60 L/min, such as during an initial purge procedure. Subsequent to an initial purge procedure, a flow rate of at least 5 L/min, at least 10 L/min, at least 20 L/min or at least 30 L/min can be maintained, such as to maintain a constant humidity level (e.g. a relative humidity between 20% and 24%). Chamber 60 can be further constructed and arranged to control temperature, such as to control temperature within chamber 60 to a temperature between 15° C. and 25° C., such as between 16° C. and 20° C. with a relative humidity between 20% and 24%. In some embodiments, one or more objects or surfaces within chamber 60 are constructed of an electrically insulating material and/or do not include sharp edges or exposed electrical components. In some embodiments, one or more metal objects positioned within chamber 60 are electrically grounded and/or maintained at a particular desired voltage level (e.g. a voltage level different than the voltage level of nozzle 427 and/or different than the voltage level of mandrel 250.

In some embodiments, system 10 is configured to produce a graft device 100′ based on one or more component or process parameters. In these embodiments, graft device 100′ comprises tubular conduit 120′ and a fiber matrix 110′ applied by electrospinning device 400. Fiber matrix 110′ can be applied via nozzle assembly 405 supplied with polymer material 111 at a flow rate of approximately 15 ml/hr. Fiber matrix 110′ can be applied when an electrostatic potential of approximately 17 kV is applied between nozzle 427 and mandrel 250, such as when nozzle 427 is charged to a potential of approximately +15 kV and mandrel 250 is charged to a potential of approximately −2 kV. Cumulative application time of fiber matrix 110′ can comprise an approximate time period of between 11 minutes and 40 seconds and 17 minutes and 30 seconds. The cumulative application time of fiber matrix 110′ can comprise a time period of approximately 11 minutes and 40 seconds when tubular conduit 120′ comprises an outer diameter of between approximately 3.4 mm and 4.2mm, a time period of approximately 14 minutes and 0 seconds when tubular conduit 120′ comprises an outer diameter between approximately 4.2 mm and 5.1 mm, and/or a time period of approximately 17 minutes and 30 seconds when tubular conduit 120′ comprises an outer diameter between approximately 5.1 mm and 6.0 mm.

Fiber matrix 110′ can comprise an average fiber size of approximately 7.8 μm, such as a population of fiber diameters with an average fiber size of approximately 7.8 μm with a standard deviation of 0.45 μm. Fiber matrix 110′ can comprise an average porosity of approximately 50.4%, such as a range of porosities with an average of 50.4% and a standard deviation of 1.1%. Fiber matrix 110′ can comprise a strength property selected from the group consisting of: stress measured at 5% strain comprising between 0.4 MPa and 1.1 MPa; ultimate stress of 4.5 MPa to 7.0 MPa; ultimate strain of 200% to 400%; and combinations of these. Fiber matrix 110′ can comprise a compliance between approximately 0.2×10⁻⁴/mmHg and 3.0×10⁻⁴/mmHg when measured in arterial pressure ranges. Fiber matrix 110′ can comprise an elastic modulus between 10 MPa and 15 MPa. Fiber matrix 110′ can be constructed and arranged with a targeted suture retention strength, such as an approximate suture retention strength of between 2.0N and 4.0N with 6-0 Prolene™ suture and/or between 1.5N and 3.0N with 7-0 Prolene™ suture. In some embodiments, graft device 100′ includes a spine 210′, such as a spine 210′ placed between inner and outer layers of fiber matrix 110′ (e.g. placed after one-third of the total thickness of fiber matrix 110′ is applied about conduit 120′).

In some embodiments, system 10 is configured to produce a graft device 100″ based on one or more component or process parameters. In these embodiments, graft device 100″ comprises tubular conduit 120″ and a fiber matrix 110″ applied by electrospinning device 400. Fiber matrix 110″ can be applied via nozzle assembly 405 supplied with polymer material 111 at a flow rate of approximately 20 ml/hr. Fiber matrix 110″ can be applied when an electrostatic potential of approximately 19 kV is applied between nozzle 427 and mandrel 250, such as when nozzle 427 is charged to a potential of approximately +17 kV and mandrel 250 is charged to a potential of approximately −2 kV. Cumulative application time of fiber matrix 110″ can comprise an approximate time period of between 9 minutes and 30 seconds and 13 minutes and 40 seconds. The cumulative application time of fiber matrix 110″ can comprise a time period of approximately 9 minutes and 30 seconds when tubular conduit 120″ comprises an outer diameter between approximately 3.4 mm and 4.2 mm; a time period of approximately 11 minutes and 30 seconds when tubular conduit 120″ comprises an outer diameter between approximately 4.2 mm and 5.1 mm, and/or a time period of approximately 13 minutes and 40 seconds when tubular conduit 120″ comprises an outer diameter between approximately 5.2 mm and 6.0 mm.

Fiber matrix 110″ can comprise an average fiber size of approximately 8.6 μm, such as a population of fiber diameters with an average fiber size of approximately 8.6 μm with a standard deviation of 0.45 μm. Fiber matrix 110″ can comprise an average porosity of approximately 46.9%, such as a range of porosities with an average of 46.9% and a standard deviation of 0.9%. Fiber matrix 110″ can comprise a strength property selected from the group consisting of: stress at 5% strain comprising between 0.6 MPa and 1.3 MPa; ultimate stress of 5.0 MPa to 7.5 MPa; ultimate strain of 200% to 400%; and combinations of these. Fiber matrix 110″ can comprise an average compliance (hereinafter “compliance) between approximately 0.2×10⁻⁴/mmHg and 3.0×10⁻⁴/mmHg when measured in arterial pressure ranges. Fiber matrix 110” can comprise an elastic modulus between 12 MPa and 18 MPa. Fiber matrix 110″ can be constructed and arranged with a targeted suture retention strength, such as an approximate suture retention strength of between 2.3N and 4.3N with 6-0 Prolene™ suture and/or between 2.0N and 3.5N with 7-0 Prolene™ suture. In some embodiments, graft device 100″ includes a spine 210″, such as a spine 210″ placed between inner and outer layers of fiber matrix 110″ (e.g. placed after one-third of the total thickness of fiber matrix 110″ is applied about conduit 120″).

Fiber matrix 110′ and 110″ can comprise one or more similar features and/or one or more dissimilar features. Fiber matrix 110″ of graft device 100″ can comprise more bonds between fibers than fiber matrix 110′ of graft device 100′. The increased number of bonds can result in a higher fiber matrix 110″ density which can be configured to limit cellular infiltration into graft device 100″ (e.g. to increase the graft durability in vivo). Fiber matrix 110″ can comprise fibers that are flatter (i.e. more oval versus round) and/or denser than fibers of fiber matrix 110′. Fiber matrix 110″ can have a greater resiliency than fiber matrix 110′.

Referring now to FIG. 4, a side sectional view of a portion of electrospinning device 400 of FIG. 3 is illustrated, consistent with the present inventive concepts. Electrospinning device 400 includes nozzle assembly 405 as has been described hereabove. Nozzle assembly 405 is operably attached to linear drive assembly 445, such as to allow reciprocating motion (in and out of the page). Nozzle assembly 405 includes nozzle 427, which is positioned in (e.g. fixed to) sleeve 406. Nozzle 427 is fluidly attached to delivery tube 425, such as to receive polymer material 111 of FIG. 3. Nozzle 427 is connected to a power supply, not shown but such as power supply 410 described hereabove in reference to FIG. 3. Surrounding nozzle 427 is a tube, sheath 407, which can also be positioned in (e.g. fixed to) sleeve 406. In some embodiments, a relatively continuous separation, gap 408, is positioned between the inner surface of sheath 407 and the outer surface of nozzle 427. Also shown in FIG. 4 is mandrel 250, which is surrounded by (e.g. has been inserted into) tubular conduit 120. At least a portion of a fiber matrix, inner layer 110a, has been applied circumferentially about tubular conduit 120. In a subsequent step, a spine or other kink resisting element can be applied about at least a portion of inner layer 110a, and/or an outer layer of fiber matrix can be applied about all or a portion of inner layer 110a.

In some embodiments, sleeve 406 is made of an electrically non-conductive material, such as an electrically non-conductive plastic such as polyoxymethylene (POM). Sleeve 406 can be constructed of electrically non-conductive materials to electrically isolate one or more components of polymer delivery assembly 405. Alternatively, sleeve 406 can comprise electrically conductive material, such as to apply a pre-determined electrical potential to sleeve 406 and/or to simplify electrical connection between one or more components of polymer delivery assembly 405, such as to simplify an electrical connection of nozzle 427 to a power supply of device 400. Similarly, sheath 407 can comprise an electrically conductive and/or an electrically non-conductive material. Sheath 407 can comprise a hypotube, such as a metal hypotube comprising the same material as nozzle 427 (e.g. stainless steel such as 403 stainless steel). Sheath 407 can be electrically connected with nozzle 427, such as via direct contact with nozzle 427 or via a wire, not shown. In some embodiments, a conductive sheath 407 that is electrically connected to nozzle 427 is constructed and arranged to limit inadvertent lateral motion of a delivered fiber stream and/or to reduce the likelihood of icicle formation (i.e. where the fiber streams wicks to the edge of nozzle 427 and forms potentially undesirable secondary streams of fiber). Alternatively, a non-conductive sheath 407 can be constructed and arranged to diminish the electrical field effect to the fiber stream while allowing for the collection of vapor in gap 408, which can prevent adverse effects on the stream as it spreads across the face of nozzle 427. Nozzle 427 can comprise a hypotube with a blunt distal end (e.g. a blunt end that is relatively orthogonal to the axis 428 of nozzle 427 and comprises minimal filleting or chamfering). Nozzle 427 can comprise a length of between 0.5 inches and 1.5 inches, such as a length of approximately 1.0 inches. In some embodiments, approximately 1.0 cm of nozzle 427 extends below sleeve 406. Nozzle 427 can comprise an ID between 0.014 inches and 0.018 inches, such as an ID of approximately 0.016 inches. Nozzle 427 can comprise a wall thickness of approximately 0.004 inches to 0.018 inches, such as a wall thickness of approximately 0.006 inches. In some embodiments, nozzle 427 comprises a wall with a stepped (e.g. multiple thickness) profile, such as a nozzle 427 with a thicker wall at its midsection than on its distal end.

Sheath 407 can be constructed and arranged to limit (e.g. eliminate or otherwise reduce) “icicle formation” during the electrospinning process. Icicles are secondary jets that can form from the nozzle by several phenomenons, including solidified polymer solution, trapped gas bubbles, field instabilities and/or field disuniformities. For example, icicles can include polymer solution that is suspended (e.g., dripping or hanging) from the nozzle 427. The distal end of sheath 407 can be positioned flush (e.g. aligned) with the distal end of nozzle 427 as shown. The distal end of sheath 407 can comprise an end relatively perpendicular to the axis 428 of nozzle 427, such as a sharp and/or deburred end. In some embodiments, sheath 407 comprises an ID slightly greater than the OD of nozzle 427, such as to create a gap 408. In other embodiments, sheath 407 is in contact with nozzle 427, avoiding gap 408. In yet other embodiments, sheath 407 and nozzle 427 comprise a single component (e.g. a single, thick-walled tube). Sheath 407 can comprise an ID of approximately 0.080 inches and/or an OD of approximately 0.118 inches. Sheath 407 can comprise a wall thickness of between 0.025 inches and 0.085 inches, such as a wall thickness of approximately 0.055 inches. Sheath 407 can comprise a length between 12 mm and 20 mm, such as a length of approximately 16 mm.

In some embodiments, central axis 428 of nozzle 427 is relatively vertical, and perpendicular to central axis 435 of mandrel 250. Axis 428 of nozzle 427 can be offset from axis 435 of mandrel 250, such as an offset along a horizontal plane of approximately 0.3 cm to 2.0 cm, such as an offset of 0.5 cm to 0.8 cm. This horizontal offset, offset HO1 shown, can be configured to limit (e.g. prevent) material provided to the nozzle 427 (e.g. polymer solution) from inadvertently being deposited (e.g. dripping due to gravity) onto the tubular conduit 120 or the fiber matrix 110.

In some embodiments, electrospinning device 400 includes one or more “object free zones” such as zones Z1, Z2, and Z3 shown in FIG. 4 and comprising one or more volumes of space that are absent of objects that could interfere with the electrospinning process of device 400. Zone Z1 comprises a cylindrical volume centered about axis 435 of mandrel 250. In some embodiments, zone Z1 comprises a diameter of between 5 cm and 15 cm, such as a diameter of approximately 10 cm. Zone Z1 can comprise a length approximating the length of tubular conduit 120 and/or mandrel 250. Zone Z2 comprises a cylindrical volume centered about axis 428 of nozzle 427. Zone Z2 extends below the distal end of nozzle 427 and comprises a diameter of between 5 cm and 15 cm, such as a diameter of approximately 10 cm. The object-free zones can take any shape, and can include one or more volumes of space positioned about nozzle 427 (e.g. zone Z1), about mandrel 250 (e.g. zone Z2) and/or about and including the volume of space between the surface of the distal end of nozzle 427 and the outer surface of mandrel 250 (e.g. zone Z3 as shown in FIG. 4). In some embodiments, object-free zones (e.g. Z1, Z2, and/or Z3) are sized and configured to be large enough to prevent one or more of: adversely affecting the electromagnetic field between nozzle 427 and mandrel 250; having an object interfere with (e.g. collide with) the flight path of a fiber traveling between nozzle 427 and mandrel 250; and allowing polymer material to drip onto tubular conduit 120 and/or fiber matrix 110. In these embodiments, object-free zones Z1, Z2, and/or Z3 are of a small enough size to permit adequate desired deposition of fibers onto the tubular conduit 120 and/or the fiber matrix 110 during operation of device 400.

While the graft devices herein have been described in detail as generally including an electrospun fiber matrix, other fiber delivery or other material application equipment can be used. Also, in some embodiments, the graft devices can include one or more spines or other kink resisting elements, or the applied fiber matrix can be configured to sufficiently resist kinking without the inclusion of the spine.

While some example embodiments of the systems, methods and devices have been described in reference to the environment in which they were developed, they are merely been described as such for illustrative purposes. Modification or combinations of the above-described assemblies, other embodiments, configurations, and methods, as well as other variations of the aspects described herein are intended to be within the scope of the claims. In addition, where this application has listed the example steps of a method or procedure in a specific order, it can be possible, or even expedient in some circumstances, to change the order in which some steps are performed, and it is intended that the particular steps of the method or procedure claim set forth herebelow not be construed as being order-specific unless such order specificity is expressly stated in the claim.

What is claimed:

1. A system for producing a graft device, the system comprising:
a rotating assembly constructed and arranged to rotate a tubular conduit;
a polymer delivery assembly constructed and arranged to receive a polymer and deliver a fiber matrix comprising the polymer about the tubular conduit;
a controller constructed and arranged to control the polymer delivery assembly and the rotating assembly; and
a diagnostic assembly constructed and arranged to detect an undesired state of at least one of the system or the graft device.

2. The system of any claim herein, wherein the system comprises an electrospinning system.

3. The system of any claim herein, wherein the system is constructed and arranged to correct the detected undesired state.

4. The system of any claim herein, further comprising an alarm assembly constructed and arranged to activate when the undesired state is detected by the diagnostic assembly.

5. The system of claim 4, wherein the alarm assembly comprises an alert selected from the group consisting of: audible alert; visual alert; tactile alert; and combinations thereof.

6. The system of any claim herein, wherein the diagnostic assembly is constructed and arranged to detect an undesired state of a polymer delivery assembly parameter.

7. The system of claim 6, wherein the polymer delivery assembly parameter represents the presence of a leak.

8. The system of claim 6, wherein the polymer delivery assembly parameter represents a polymer flow rate.

9. The system of claim 6, further comprising a polymer flow pathway, wherein the polymer delivery assembly parameter represents a level of undesired material in the polymer flow pathway.

10. The system of claim 9, wherein the undesired material comprises undesired particulate.

11. The system of claim 9, wherein the undesired material comprises material with an undesired homogeneity.

12. The system of claim 9, wherein the undesired material comprises a gas bubble.

13. The system of claim 9, wherein the undesired material comprises a material selected from the group consisting of: water; blood; lubricant; isopropyl alcohol; disinfectant; solvent; and combinations thereof.

14. The system of claim 6, wherein the polymer comprises an expiration date, and wherein the polymer delivery assembly parameter represents an expiration date of the polymer.

15. The system of claim 6, wherein the polymer comprises a polymer parameter, and wherein the polymer delivery assembly parameter represents a polymer parameter selected from the group consisting of: polymer viscosity; polymer conductivity; polymer surface tension; polymer color; polymer turbidity; polymer chemical composition; polymer molecular weight profile; polymer magnetism; polymer impedance; and combinations thereof.

16. The system of claim 6, wherein the polymer delivery assembly comprises a nozzle constructed and arranged to translate, and wherein the polymer delivery assembly parameter represents the translation rate of the nozzle.

17. The system of claim 6, wherein the polymer delivery assembly comprises a nozzle constructed and arranged to translate, and wherein the polymer delivery assembly parameter represents the translation acceleration of the nozzle.

18. The system of claim 6, wherein the polymer delivery assembly comprises a nozzle constructed and arranged to translate, and wherein the polymer delivery assembly parameter represents the position of the nozzle.

19. The system of claim 6, wherein the polymer delivery assembly comprises a nozzle constructed and arranged to translate, and wherein the polymer delivery assembly parameter represents the position of the nozzle relative to the rotating assembly.

20. The system of claim 6, wherein the polymer delivery assembly comprises a nozzle constructed and arranged to translate, and wherein the polymer delivery assembly parameter represents nozzle vibration level.

21. The system of claim 6, wherein the polymer delivery assembly comprises a nozzle constructed and arranged to translate, and wherein the polymer delivery assembly parameter represents status of nozzle contacting an undesired object.

22. The system of claim 6, wherein the polymer delivery assembly parameter represents a fiber parameter.

23. The system of claim 22, wherein the fiber parameter comprises a fiber parameter selected from the group consisting of: diameter; average diameter; diameter range; porosity; nodal density; alignment; flatness; twist; elasticity; crystallinity; conformity to target; water content; and combinations thereof.

24. The system of claim 22, wherein the polymer delivery assembly parameter represents a fiber flight pathway parameter.

25. The system of claim 6, wherein the polymer delivery assembly parameter represents a fiber matrix parameter.

26. The system of claim 25, wherein the fiber matrix parameter comprises a fiber matrix parameter selected from the group consisting of: porosity; thickness; density; thickness distribution along the two longitudinal and circumferential axes; and combinations thereof.

27. The system of claim 6, wherein the polymer delivery assembly comprises a nozzle, and wherein the polymer delivery assembly parameter represents a voltage level applied to the nozzle.

28. The system of claim 27, wherein the rotating assembly comprises a mandrel constructed and arranged to be slidingly received by the tubular conduit, and wherein the polymer delivery assembly parameter further represents a voltage level applied to the mandrel.

29. The system of claim 6, wherein the polymer delivery assembly comprises a nozzle, and wherein the polymer delivery assembly parameter represents the presence of icicles about the nozzle.

30. The system of any claim herein, wherein the diagnostic assembly is constructed and arranged to detect an undesired state of a rotating assembly parameter.

31. The system of claim 30, wherein the rotating assembly comprises a mandrel, and wherein the rotating assembly parameter represents rotational velocity of the mandrel.

32. The system of claim 30, wherein the rotating assembly comprises a mandrel, and wherein the rotating assembly parameter comprises a voltage level applied to the mandrel.

33. The system of claim 30, wherein the rotating assembly comprises a mandrel, and wherein the rotating assembly parameter represents alignment of the mandrel.

34. The system of any claim herein, wherein the diagnostic assembly is constructed and arranged to detect an undesired state of a controller parameter.

35. The system of claim 34, wherein the controller comprises a power supply and wherein the controller parameter represents an input level of the power supply.

36. The system of claim 34, wherein the controller comprises a power supply and wherein the controller parameter represents an output level of the power supply.

37. The system of claim 34, wherein the controller comprises at least one electrical connection and the controller parameter represents connection status of the at least one electrical connection.

38. The system of any claim herein, wherein the diagnostic assembly is constructed and arranged to detect an undesired state of a tubular conduit parameter.

39. The system of claim 38, wherein the tubular conduit parameter represents a diameter of the tubular conduit.

40. The system of claim 38, wherein the tubular conduit parameter represents level of trauma in the tubular conduit.

41. The system of claim 40, wherein the tubular conduit comprises a wall, and wherein the level of trauma represents a level of disruption in the wall of the tubular conduit.

42. The system of claim 38, wherein the tubular conduit comprises a wall, and wherein the tubular conduit parameter represents the status of a leak in the wall of the tubular conduit.

43. The system of claim 42, wherein the leak comprises a leak in an insufficiently ligated side branch of the tubular conduit.

44. The system of any claim herein, wherein the diagnostic assembly is constructed and arranged to detect an undesired state of a fiber matrix parameter.

45. The system of claim 44, wherein the fiber matrix parameter represents a thickness of the fiber matrix.

46. The system of claim 44, wherein the fiber matrix parameter represents a dryness level of the fiber matrix.

47. The system of claim 44, wherein the fiber matrix parameter represents a fiber matrix parameter selected from the group consisting of: fiber diameter; fiber average diameter; fiber diameter range; nodal density; fiber alignment; fiber flatness; fiber twist; fiber elasticity; fiber crystallinity; fiber conformity to target; fiber water content; fiber matrix porosity; fiber matrix thickness; fiber matrix density; fiber matrix thickness distribution along the longitudinal and circumferential axes; and combinations thereof.

48. The system of any claim herein wherein the graft device further comprises a spine, wherein the diagnostic assembly is constructed and arranged to detect an undesired state of a spine parameter.

49. The system of claim 48, wherein the spine parameter represents the position of the spine about the tubular conduit.

50. The system of claim 48, wherein the spine parameter comprises a spine parameter selected from the group consisting of: spine size; spine position; compression level applied to tubular conduit; and combinations thereof.

51. The system of any claim herein, wherein the diagnostic assembly is constructed and arranged to detect an undesired state of a graft device parameter.

52. The system of claim 51, wherein the graft device parameter represents a diameter of the graft device.

53. The system of claim 51, wherein the graft device parameter represents a solvent level present in the graft device.

54. The system of any claim herein, wherein the system comprises a sensor constructed and arranged to collect data used to detect the undesired state of that at least one of the system or the graft device.

55. The system of claim 54, wherein the sensor comprises a sensor selected from the group consisting of: environmental sensor; pressure sensor; strain gauge; temperature sensor; humidity sensor; vibration sensor; pH sensor; chemical sensor; solvent sensor; magnetic sensor; electromagnetic sensor; ultrasonic sensor; flow sensor; viscosity sensor; visual sensor; optical sensor; light sensor; and combinations thereof.

56. The system of claim 54, wherein the sensor comprises a viscosity sensor.

57. The system of claim 56, wherein the data collected comprises polymer viscosity data.

58. The system of claim 54, further comprising an environmental chamber surrounding at least a portion of the rotating assembly, wherein the sensor comprises an environmental parameter sensor constructed and arranged to measure an environmental parameter within the environmental chamber.

59. The system of claim 58, wherein the measured environmental parameter comprises a parameter selected from the group consisting of: temperature; humidity; pressure; and combinations thereof.

60. The system of claim 54, wherein the sensor comprises a temperature sensor.

61. The system of claim 60, further comprising a polymer storage device, wherein the data produced by the temperature sensor represents a thermal history of the polymer storage device.

62. The system of claim 60, wherein the temperature sensor is constructed and arranged to measure temperature of the polymer.

63. The system of claim 62, wherein the system is constructed and arranged to filter the polymer, and wherein the temperature sensor is constructed and arranged to measure the temperature of the polymer during filtration.

64. The system of claim 54, wherein the sensor comprises a leak-detecting sensor.

65. The system of claim 64, wherein the leak sensor comprises a fluid-detecting sensor.

66. The system of claim 64, wherein the leak sensor comprises a pressure sensor.

67. The system of claim 54, wherein the sensor comprises a polymer solution homogeneity sensor.

68. The system of claim 67, wherein the polymer solution homogeneity sensor comprises a light sensor.

69. The system of claim 54, wherein the sensor comprises at least one of a motion sensor or a position sensor.

70. The system of claim 69, wherein the at least one of a motion sensor or a position sensor comprises a sensor selected from the group consisting of: optical; magnetic; and combinations thereof.

71. The system of claim 69, wherein the at least one of a motion sensor or a position sensor is constructed and arranged to detect an undesired translation of the polymer delivery assembly.

72. The system of claim 69, wherein the rotating assembly further comprises a mandrel, and wherein the at least one of a motion sensor or a position sensor is constructed and arranged to detect undesired rotation of the mandrel.

73. The system of claim 54, wherein the sensor comprises a voltage sensor.

74. The system of claim 73, wherein the voltage sensor is constructed and arranged to detect voltage of the polymer delivery assembly.

75. The system of claim 74, wherein the polymer delivery assembly comprises a nozzle, and wherein the voltage sensor is constructed and arranged to detect voltage of the nozzle.

76. The system of claim 73, wherein the rotating assembly comprises a mandrel, and wherein the voltage sensor is constructed and arranged to detect voltage of the mandrel.

77. The system of claim 54, wherein the sensor comprises an image producing sensor.

78. The system of claim 77, wherein the image producing sensor comprises a camera.

79. The system of claim 77, further comprising an image processing algorithm constructed and arranged to analyze the data produced by the image producing sensor, wherein the detection of the undesired state is based on the analysis.

80. The system of claim 77, wherein the polymer delivery assembly comprises a nozzle, and wherein the image producing sensor is constructed and arranged to provide visual information related to fibers delivered by the nozzle.

81. The system of claim 80, wherein the nozzle is constructed and arranged to produce a Taylor Cone proximate the nozzle tip, and wherein the image producing sensor is constructed and arranged to provide visual information related to the Taylor Cone.

82. The system of claim 80, wherein the image producing sensor is constructed and arranged to provide visual information related to a fiber parameter selected from the group consisting of: fiber transparency; fiber translucency; fiber diameter; and combinations thereof.

83. The system of claim 80, wherein the image producing sensor is constructed and arranged to provide visual information related to any undesired objects proximate the nozzle.

84. The system of claim 54, wherein the sensor comprises a measurement sensor.

85. The system of claim 84, wherein the measurement sensor comprises a visual sensor.

86. The system of claim 85, wherein the visual sensor comprises a camera.

87. The system of claim 84, wherein the measurement sensor comprises an optical sensor.

88. The system of claim 87, wherein the optical sensor comprises a laser.

89. The system of claim 84, wherein the measurement sensor comprises a surface-detecting sensor.

90. The system of claim 89, wherein the surface-detecting sensor comprises a sensor selected from the group consisting of: light sensor; radar sensor; sonar sensor; and combinations thereof.

91. The system of claim 84, wherein the sensor is constructed and arranged to measure a fiber matrix property.

92. The system of claim 91, wherein the fiber matrix property comprises a fiber matrix property selected from the group consisting of: fiber diameter; fiber average diameter; fiber diameter range; nodal density; fiber alignment; fiber flatness; fiber twist; fiber elasticity; fiber crystallinity; fiber conformity to target; fiber water content; fiber matrix porosity; fiber matrix thickness; fiber matrix density; fiber matrix thickness distribution along the longitudinal and circumferential axes; and combinations thereof.

93. The system of claim 54, wherein the sensor comprises a flow sensor.

94. The system of claim 93, wherein the flow sensor is constructed and arranged to measure a polymer flow rate.

95. The system of claim 93, further comprising an environmental chamber surrounding at least a portion of the rotating assembly, wherein the flow sensor is constructed and arranged to measure the flow rate of gas supplied to the environmental chamber.

96. The system of claim 93, further comprising an environmental chamber surrounding at least a portion of the rotating assembly, wherein the flow sensor is constructed and arranged to measure the flow rate of gas evacuated from the environmental chamber.

97. The system of claim 93, further comprising an environmental control chamber surrounding at least a portion of the polymer delivery assembly, and wherein the flow sensor is constructed and arranged to measure the flow rate of a gas within the environmental control chamber.

98. The system of claim 93, wherein the polymer delivery assembly comprises a nozzle, and wherein the flow sensor is constructed and arranged to measure the flow rate into the nozzle.

99. The system of claim 54, wherein the sensor comprises an occlusion sensor.

100. The system of claim 99, wherein the system further comprises at least one polymer flow pathway and wherein the occlusion sensor is constructed and arranged to measure flow in the at least one polymer flow pathway.

101. The system of claim 54, wherein the sensor comprises a sensor constructed and arranged to measure a contamination level.

102. The system of claim 101, wherein the system further comprises at least one polymer flow pathway and wherein the contamination sensor is constructed and arranged to measure contamination level in the at least one polymer flow pathway.

103. The system of claim 101, wherein the contamination sensor is constructed and arranged to measure contamination level in and/or on the fiber matrix.

104. The system of claim 101, wherein the contamination sensor is constructed and arranged to measure contamination level in and/or on the tubular conduit.

105. The system of claim 54, wherein the sensor comprises a sensor constructed and arranged to measure a level of solvent.

106. The system of claim 105, wherein the sensor comprises a sensor selected from the group consisting of: colorimetric detector tube; passive (diffusion) badge dosimeter; sorbent tube sampling device; combustible gas monitor such as a monitoring using a hot bead or a hot wire; combustible gas sensor; photoionization detector; flame ionization detector; infrared spectra-photometer; and combinations thereof.

107. The system of claim 105, further comprising an environmental chamber surrounding at least a portion of the rotating assembly and a filter on an outflow port of the environmental chamber, wherein the sensor is constructed and arranged to measure a parameter of the outflow port filter.

108. The system of claim 107, wherein the sensor is constructed and arranged to measure a parameter selected from the group consisting of: weight of the outflow port filter; flow through the outflow port filter; and combinations thereof.

109. The system of any claim herein, further comprising an information element and an information element reader device constructed and arranged to collect data from the information element, wherein the diagnostic assembly analyzes the collected data to detect the undesired state of at least one of the system or the graft device.

110. The system of claim 109, wherein the information element comprises an element selected from the group consisting of: barcode; microchip; RFID; and combinations thereof.

111. The system of claim 109, further comprising a polymer storage device comprising the information element, wherein the information element data comprises polymer data.

112. The system of claim 111, wherein the diagnostic assembly detects the applicability of the polymer based on the polymer data.

113. The system of claim 111, wherein the diagnostic assembly detects an expiration date of the polymer based on the polymer data.

114. The system of any claim herein, further comprising a timer constructed and arranged to measure the time period of delivery of the fiber matrix to the tubular conduit.

115. The system of claim 114, wherein the undesired state detected by the diagnostic assembly comprises a measured time period of delivery below a minimum.

116. The system of claim 114, wherein the undesired state detected by the diagnostic assembly comprises a measured time period of delivery above a maximum.

117. A method of producing a graft device, the method comprising:
selecting a system of any claim herein; and
applying a fiber matrix about a tubular conduit.

118. The method of any claim herein, further comprising entering an alarm state when an undesired state of at least one of the system or graft device is detected.

119. The method of claim 118, wherein entering the alarm state comprises producing an alert signal.

120. The method of claim 119, wherein the alert signal comprises a signal selected from the group consisting of: audible alert; visual alert; tactile alert; and combinations thereof.

121. The method of claim 118, wherein entering the alarm state comprises stopping the delivery of the fiber matrix about the tubular conduit.

122. A system as described in reference to the drawings.

123. A method as described in reference to the drawings.
